(19) United States
(12) Patent Application Publication
(10) Pub. No.: US 2025/0041592 A1
(43) Pub. Date: Feb. 6, 2025

(54) SYSTEMS AND DEVICES FOR RECORDING NEURAL ACTIVITY OR STIMULATING NEURAL TISSUE AND METHODS OF OPERATION THEREOF

(71) Applicants: Nicholas Lachlan OPIE, Melbourne (AU); Stephen Michael RONAYNE, Parkville (AU); Michael HAINES, Ormond (AU); Gil Simon RIND, Northcote (AU)

(72) Inventors: Nicholas Lachlan OPIE, Melbourne (AU); Stephen Michael RONAYNE, Parkville (AU); Michael HAINES, Ormond (AU); Gil Simon RIND, Northcote (AU)

(21) Appl. No.: 18/792,965

(22) Filed: Aug. 2, 2024

Related U.S. Application Data
(60) Provisional application No. 63/517,495, filed on Aug. 3, 2023.

(51) Int. Cl. A61N 1/05; A61N 1/36; A61N 1/372

(52) U.S. Cl. CPC A61N 1/0539; A61N 1/3605; A61N 1/372

(57) ABSTRACT

Disclosed herein are systems, devices, and methods for implanting electrodes within a cerebral vessel of a subject. For example, disclosed are various types of implantable electrode arrays, stents carrying electrode arrays, delivery devices for implantable electrode arrays, electrical interfaces for an implantable electrode array, connector lead cable assemblies for an implantable electrode array, and methods of delivering and deploying implantable electrode arrays.

### CROSS-REFERENCE TO RELATED APPLICATION

This application claims the benefit of U.S. Provisional Application No. 63/517,495 filed on Aug. 3, 2023, the content of which is incorporated herein by reference in its entirety.

### FIELD OF TECHNOLOGY

The present disclosure relates generally to the field of medical devices for implantation in the brain, and, more specifically, to systems and devices for recording neural activity and/or stimulating neural tissue and methods of operation thereof.

### BACKGROUND

Millions of people around the world suffer from various neuromuscular or neurological disorders or diseases where control of limbs is severely impaired or limited. For these people, the ability to restore lost control, at even a rudimentary level, could lead to a greatly improved quality of life. One option for restoring function to such individuals is a brain computer interface (BCI) system. Such systems often include one or more implantable devices for recording a subject's neural activity and, in some cases, the same implantable devices or an additional implantable device can be used to stimulate the subject's neural tissue.

However, current BCI systems rely on implantable devices that may not be appropriate for implantation or deployment in smaller cerebral vessels that often branch out from a larger cerebral vessel such as the superior sagittal sinus. Such vessels are often less than 4 mm in diameter or even less than 2 mm in diameter and are also more delicate. Moreover, such vessels can also be more tortuous than their larger counterparts.

Therefore, a solution is needed for a BCI system with one or more implantable devices that can be permanently implanted or deployed into smaller cerebral vessels. These devices should be able to navigate tortuous anatomy, be delivered and deployed without adversely affecting blood flow, provide for proper recording and/or stimulation via electrical contacts, and be securable within a target site to enhance apposition with the vessel and to prevent migration. Such devices should also be able to access branched vessels and allow the BCI system to access a larger portion of the motor homunculus and the somatosensory regions of the brain for purposes of recording or stimulating neural tissue.

### SUMMARY

Disclosed are systems, devices, and methods for implanting electrodes within a cerebral vessel of a subject. In one embodiment, a system for implanting electrodes within a cerebral vessel of a subject can comprise a microwire, and a tubular member comprising a tubular body sized to fit within the cerebral vessel, a central lumen extending through the tubular body, a plurality of peripheral lumens extending through the tubular body, a plurality of wires extending through each of the peripheral lumens; and a plurality of electrodes coupled to the tubular body. The central lumen can be sized to be advanced over the microwire. The plurality of peripheral lumens can surround the central lumen. The plurality electrodes can be spaced apart along a length of the tubular body. A conductive portion of one of the wires can be connected to one of the electrodes.

In some embodiments, the electrodes can be cylindrical electrodes or ring electrodes such that each of the cylindrical electrodes or ring electrodes circumferentially surround the tubular body.

In some embodiments, the electrodes can be cuff electrodes such that each of the cuff electrodes partially surround a circumference of the tubular body but does not completely surround the tubular body.

In some embodiments, at least some of the cuff electrodes can be circumferentially misaligned with respect to one another along part of the length of the tubular body.

In some embodiments, the electrodes can be semicircular electrodes such that each of the semicircular electrodes surround half of a circumference of the tubular body.

In some embodiments, at least some of the semicircular electrodes can be circumferentially offset with respect to one another along part of the length of the tubular body.

In some embodiments, the electrodes can be positioned in a helical pattern along the length of the tubular body. A pitch of the helical pattern can be between about 2.0 mm and about 4.0 mm.

In some embodiments, the plurality of wires extending through each of the peripheral lumen can comprise between two wires and seven wires.

In some embodiments, at least a segment of each of the wires can be coated by an insulating layer.

In some embodiments, a wire diameter of at least one of the wires can be between 40 μm to 60 μm.

In some embodiments, a diameter of the central lumen can be between 300 μm to 500 μm.

In some embodiments, a diameter of at least one of the peripheral lumens can be between 100 μm to 200 μm.

In some embodiments, the plurality of peripheral lumens can radially surround the central lumen.

In some embodiments, a radially outermost point of each of the peripheral lumens furthest from a peripheral lumen center can be separated from an exterior surface of the tubular body by at least 25 μm.

In some embodiments, a radially innermost point of each of the peripheral lumens furthest from a peripheral lumen center can be separated from a radially outermost point of the central lumen further from a central lumen center by at least 25 μm.

In some embodiments, an outer diameter of the tubular body can be between 600 μm to 800 μm.

In some embodiments, a total length of the tubular body can be between 80 mm and 120 mm.

In some embodiments, each of the electrodes can be spaced apart from its nearest neighboring electrode by about 2.0 mm to about 4.0 mm.

In some embodiments, the plurality of peripheral lumens can comprise between four peripheral lumens and eight peripheral lumens.

In some embodiments, the plurality of electrodes can comprise between 32 electrodes and 56 electrodes.

In some embodiments, the system can further comprise a pre-shaped helical wire sized to extend through the central lumen of the tubular body when the microwire is removed from the central lumen. The pre-shaped helical wire can be configured to force at least part of the tubular body to attain a helical or coiled configuration when the pre-shaped helical wire is extended through at least part of the central lumen.

In some embodiments, the tubular body can be configured to self-coil into a helical or coiled configuration in response to the microwire being removed from the central lumen of the tubular member. The plurality of electrodes can be configured to press against a vessel wall of the cerebral vessel when the tubular body is in the helical or coiled configuration.

In some embodiments, the system can further comprise an anchoring wire comprising a hooked portion at a distal end of the anchoring wire. The anchoring wire can be sized to extend through the central lumen of the tubular body when the microwire can be removed from the central lumen. The hooked portion of the anchoring wire can be configured to extend distally out of the central lumen to secure the tubular member within the cerebral vessel.

In some embodiments, the system can further comprise an anchoring wire comprising: a wire distal end, an anchoring segment, and a proximal wire segment located proximal to the anchoring segment. The anchoring wire can be sized to extend through one of the peripheral lumens or another lumen extending through the tubular body. The distal end of the anchoring wire can be coupled to a distal point along a tubular body wall of the tubular member. At least part of the anchoring segment of the anchoring wire can be configured to extend through an opening defined along the tubular body wall of the tubular body. The opening can be positioned proximal to the distal point. At least part of the anchoring segment can be configured to form a looped segment outside of the tubular body when part of the proximal wire segment can be advanced distally and the looped segment can be configured to secure the tubular member within the cerebral vessel.

In some embodiments, at least one of the electrodes can comprise an electrode body converging at a sharp point to enable the electrode to penetrate through a vessel wall of the cerebral vessel when the electrode is pressed against the vessel wall.

In some embodiments, the electrode body can house a medicament or pharmaceutical and the electrode body can be configured to release the medicament or pharmaceutical when the electrode is pressed against the vessel wall.

In some embodiments, at least one of the electrodes can comprise an electrode base. The electrode can comprise a plurality of electrode bodies. Each of the electrode bodies can converge at a sharp point to enable the electrode to penetrate through a vessel wall of the cerebral vessel when the electrode is pressed against the vessel wall.

In some embodiments, the electrode base can be substantially flat.

In some embodiments, the electrode base can be curved.

In some embodiments, the plurality of electrodes can comprise a first electrode and a second electrode. The first electrode can comprise a first electrode body converging at a first sharp point to enable the first electrode to penetrate through a vessel wall of the cerebral vessel when the first electrode is pressed against the vessel wall. The second electrode can comprise a second electrode body converging at a second sharp point to enable the second electrode to penetrate through the vessel wall of the cerebral vessel when the second electrode is pressed against the vessel wall.

In some embodiments, the first electrode can have a first electrode height and the second electrode can have a second electrode height. The first electrode height can be greater than the second electrode height.

In some embodiments, the first electrode and the second electrode can be connected to the conductive portion of the same wire.

In some embodiments, the first electrode can be connected to the conductive portion of a first wire of the plurality of wires. The second electrode can be connected to the conductive portion of a second wire of the plurality of wires.

In some embodiments, the first wire and the second wire can extend through the same peripheral lumen.

In some embodiments, the first wire and the second wire can extend through different peripheral lumens.

Also disclosed is a device for implantation within a cerebral vessel of a subject. The device can comprise a tubular body sized to fit within the cerebral vessel; a central lumen extending through the tubular body; a plurality of peripheral lumens extending through the tubular body; a plurality of wires extending through each of the peripheral lumens; and a plurality of electrodes coupled to the tubular body. The plurality of peripheral lumens can surround the central lumen. The plurality electrodes can be spaced apart along a length of the tubular body. A conductive portion of one of the wires can be connected to one of the electrodes.

Also disclosed is a device for implantation within a cerebral vessel of a subject. The device can comprise a tubular body sized to fit within the cerebral vessel; a plurality of lumens extending through the tubular body; a plurality of wires extending through each of the lumens; and a plurality of electrodes coupled to the tubular body. The plurality electrodes can be spaced apart along a length of the tubular body. A conductive portion of one of the wires can be connected to one of the electrodes.

In some embodiments, the electrodes can be cylindrical electrodes or ring electrodes such that each of the cylindrical electrodes or ring electrodes circumferentially surround the tubular body.

In some embodiments, the electrodes can be cuff electrodes such that each of the cuff electrodes can partially surround a circumference of the tubular body but does not completely surround the tubular body.

In some embodiments, at least some of the cuff electrodes can be circumferentially misaligned with respect to one another along part of the length of the tubular body.

In some embodiments, the electrodes can be semicircular electrodes such that each of the semicircular electrodes surround half of a circumference of the tubular body.

In some embodiments, at least some of the semicircular electrodes can be circumferentially offset with respect to one another along part of the length of the tubular body.

In some embodiments, the electrodes can be positioned in a helical pattern along the length of the tubular body. A pitch of the helical pattern can be between about 2.0 mm and about 4.0 mm.

In some embodiments, the plurality of wires extending through each of the lumens can comprise between two wires and seven wires.

In some embodiments, at least a segment of each of the wires can be coated by an insulating layer.

In some embodiments, a wire diameter of at least one of the wires can be between 40 μm to 60 μm.

In some embodiments, a diameter of at least one of the lumens can be between 100 μm to 200 μm.

In some embodiments, the plurality of lumens can comprise a radially outer lumen. A radially outermost point of the radially outer lumen furthest from a lumen center of the radially outer lumen can be separated from an exterior surface of the tubular body by at least 25 μm.

In some embodiments, each of the lumens can be separated from its closest neighboring lumen by at least 25 μm.

In some embodiments, an outer diameter of the tubular body can be between 500 μm to 600 μm.

In some embodiments, a total length of the tubular body can be between 80 mm and 120 mm.

In some embodiments, each of the electrodes can be spaced apart from its nearest neighboring electrode by about 2.0 mm to about 4.0 mm.

In some embodiments, the plurality of lumens can comprise between three and seven lumens.

In some embodiments, the plurality of electrodes can comprise between 12 electrodes and 49 electrodes.

In some embodiments, at least one of the electrodes can comprise an electrode body converging at a sharp point to enable the electrode to penetrate through a vessel wall of the cerebral vessel when the electrode is pressed against the vessel wall.

In some embodiments, the electrode body can house a medicament or pharmaceutical. The electrode body can be configured to release the medicament or pharmaceutical when the electrode is pressed against the vessel wall.

In some embodiments, at least one of the electrodes can comprise an electrode base. The electrode can comprise a plurality of electrode bodies. Each of the electrode bodies converge at a sharp point to enable the electrode to penetrate through a vessel wall of the cerebral vessel when the electrode is pressed against the vessel wall.

In some embodiments, the electrode base can be substantially flat.

In some embodiments, the electrode base can be curved.

In some embodiments, the plurality of electrodes can comprise a first electrode and a second electrode. The first electrode can comprise a first electrode body converging at a first sharp point to enable the first electrode to penetrate through a vessel wall of the cerebral vessel when the first electrode is pressed against the vessel wall. The second electrode can comprise a second electrode body converging at a second sharp point to enable the second electrode to penetrate through the vessel wall of the cerebral vessel when the second electrode is pressed against the vessel wall.

In some embodiments, the first electrode can have a first electrode height and the second electrode has a second electrode height. The first electrode height can be greater than the second electrode height.

In some embodiments, the first electrode and the second electrode can be connected to the conductive portion of the same wire.

In some embodiments, the first electrode can be connected to the conductive portion of a first wire of the plurality of wires. The second electrode can be connected to the conductive portion of a second wire of the plurality of wires.

In some embodiments, the first wire and the second wire can extend through the same lumen.

In some embodiments, the first wire and the second wire can extend through different lumens.

Also disclosed is a method of implanting electrodes within a cerebral vessel of a subject. The method can comprise delivering a microwire to a target site in a cerebral vessel and advancing a tubular member over the microwire to the target site. The tubular member can comprise a tubular body and a central lumen extending through the tubular body. The central lumen can be sized to be advanced over the microwire. The tubular member can also comprise a plurality of peripheral lumens extending through the tubular body. The plurality of peripheral lumens can surround the central lumen and a plurality of wires can extend through each of the peripheral lumens. The tubular member can also comprise a plurality of electrodes coupled to the tubular body. The plurality electrodes can be spaced apart along a length of the tubular body. A conductive portion of each of the wires can be connected to one of the electrodes. The method can also comprise removing the microwire when the tubular member is secured at the target site.

In some embodiments, the cerebral vessel can be a vein of Labbe.

In some embodiments, the cerebral vessel can be a vein of Trolard.

In some embodiments, the cerebral vessel can be a Rolandic vein.

In some embodiments, the cerebral vessel can be a middle meningeal artery (MMA).

In some embodiments, the cerebral vessel can be less than 1.0 mm in diameter.

In some embodiments, the method can further comprise extending a pre-shaped helical wire through the central lumen of the tubular body after the microwire is removed from the central lumen. The pre-shaped helical wire can be configured to force at least part of the tubular body to attain a helical or coiled configuration when the pre-shaped helical wire is extended through at least part of the central lumen. The plurality of electrodes can be configured to press against a vessel wall of the cerebral vessel when the tubular body is in the helical or coiled configuration.

In some embodiments, the tubular body can be configured to self-coil into a helical or coiled configuration in response to the microwire being removed from the central lumen of the tubular member. The plurality of electrodes can be configured to press against a vessel wall of the cerebral vessel when the tubular body is in the helical or coiled configuration.

In some embodiments, the method can further comprise extending an anchoring wire comprising a hooked portion at a distal end of the anchoring wire through the central lumen of the tubular body when the microwire is removed from the central lumen. The hooked portion of the anchoring wire can be configured to extend distally out of the central lumen to secure the tubular member within the cerebral vessel.

In some embodiments, the tubular member can further comprise an anchoring wire comprising: a wire distal end, an anchoring segment, and a proximal wire segment located proximal to the anchoring segment. The anchoring wire can be sized to extend through one of the peripheral lumens or another lumen extending through the tubular body. The distal end of the anchoring wire can be coupled to a distal point along a tubular body wall of the tubular member. At least part of the anchoring segment of the anchoring wire can be configured to extend through an opening defined along the tubular body wall of the tubular body. The opening can be positioned proximal to the distal point. The method can further comprise distally advancing the proximal wire segment of the anchoring wire to cause the anchoring segment to form a looped segment outside of the tubular body. The looped segment can be configured to secure the tubular member within the cerebral vessel.

In some embodiments, at least one of the electrodes can comprise an electrode body converging at a sharp point to enable the electrode to penetrate through a vessel wall of the cerebral vessel when the electrode is pressed against the vessel wall.

In some embodiments, the electrode body can house a medicament or pharmaceutical. The electrode body can be configured to release the medicament or pharmaceutical when the electrode is pressed against the vessel wall.

Also disclosed is a stent. The stent can comprise a stent body comprising a plurality of struts coupled together with strut crosslinks and a plurality of electrodes coupled along the stent body. One of the struts can comprise a base layer made in part of a shape memory alloy, a first insulating layer disposed on top of at least part of the base layer, a first conductive track disposed on top of at least part of the first insulating layer, a second insulating layer disposed on top of at least part of the first conductive track, a second conductive track disposed on top of at least part of the second insulating layer, and a third insulating layer disposed on top of at least part of the second conductive track. The first conductive track can be electrically coupled to one of the electrodes. The second conductive track can be electrically coupled to another one of the electrodes,

In some embodiments, the stent can further comprise a third conductive track disposed on top of at least part of the third insulating layer. The third conductive track can be electrically coupled to yet another one of the electrodes. The stent can further comprise a fourth insulating layer disposed on top of at least part of the third conductive track.

In some embodiments, the electrodes can be coupled to electrode mounting sites along the stent body. One of the electrode mounting sites can comprise the base layer made in part of the shape memory alloy. The first insulating layer can be disposed on top of at least part of the base layer. The first conductive track can be disposed on top of at least part of the first insulating layer. The first conductive track can be electrically coupled to the electrode coupled to the electrode mounting site. The second insulating layer can be disposed on top of at least part of the first conductive track. The second conductive track can be disposed on top of at least part of the second insulating layer. The second conductive track can be electrically coupled to another one of the electrodes coupled to another one of the electrode mounting sites. The third insulating layer can be disposed on top of at least part of the second conductive track.

In some embodiments, at least part of the stent body can be in the shape of a tubular lattice or interwoven mesh structure.

In some embodiments, the base layer can be made in part of a nickel-titanium alloy.

In some embodiments, the base layer can have a thickness of between about 40 μm to about 60 μm.

In some embodiments, at least one of the first insulating layer, the second insulating layer, and the third insulating layer can be an yttrium stabilized zirconia (YSZ) layer.

In some embodiments, at least one of the first insulating layer, the second insulating layer, and the third insulating layer can have a thickness of between about 200 nm and about 400 nm.

In some embodiments, at least one of the first conductive track and the second conductive track can be made in part of platinum.

In some embodiments, at least one of the first conductive track and the second conductive track can have a thickness of between about 200 nm and about 400 nm.

In some embodiments, at least one of the first conductive track and the second conductive track can have a width of at least about 30 μm.

In some embodiments, at least one of the first conductive track and the second conductive track can be separated from an edge of the strut by at least 27 μm.

In some embodiments, at least one of the first conductive track and the second conductive track can have a track cross-section. The track cross-section can be substantially shaped as a trapezoid.

Also disclosed is a device for implanting electrodes within a cerebral vessel of a subject. The device can comprise a tubular member comprising a tubular body sized to fit within the cerebral vessel and a body distal end, and a lumen extending through the tubular body, a plurality of wires extending through the lumen. Each of the wires can comprise a distal wire segment and a distal wire end serving as a distal terminus of the wire. The distal wire segment of each of the wires can protrude out of the lumen beyond the body distal end. An electrode can be coupled to the distal wire end of each of the wires.

In some embodiments, the plurality of wires can comprise a first wire comprising a first distal wire segment having a first distal wire segment length; and a second wire comprising a second distal wire segment having a second distal wire segment length. The first distal wire segment length can be greater than the second distal wire segment length.

In some embodiments, each of the wires can comprise an inner conductive wire surrounded by an insulating coating or layer. The electrode can be electrically coupled to the inner conductive wire at the distal wire end.

In some embodiments, at least part of the distal wire segments can be unbraided or unwound such that the unbraided or unwound distal wire segments appear as elongated fingers.

Also disclosed is a method of delivering a neurovascular device within a patient. The method can comprise pre-setting a curved section of a lead device at a predetermined dimension and a predetermined shape. The curved section can comprise one or more electrodes. The method can further comprise advancing the lead device via a catheter and over a guidewire to a target location in a cerebral vessel. The curved section can straighten out via material stress during advancement. The method can further comprise advancing the lead device out of the catheter once the catheter is at the target location. The curved section can be configured to deform to its predetermined dimension and predetermined shape to produce an outward radial force on the cerebral vessel to anchor the lead device in place. The method can also comprise stimulating the cerebral vessel with the one or more electrodes.

Also disclosed is an implantable electrode array comprising an implantable carrier and an electrical interface. The implantable carrier can comprise a carrier body comprising a carrier proximal end and a carrier distal end, a plurality of electrodes coupled to the carrier body, and a paddle extending from the carrier proximal end of the carrier body. The electrical interface can comprise a flexible printed circuit coupled to the paddle of the implantable carrier and one or more conductive wires coupled to the flexible printed circuit.

In some embodiments, the flexible printed circuit can comprise a plurality of layers stacked on top of one another.

In some embodiments, the flexible printed circuit can comprise a plurality of signal layers separated by dielectric layers. At least one of the signal layers can comprise conductive traces.

In some embodiments, the signal layers can comprise a top-most signal layer and a bottom-most signal layer. The top-most signal layer can be covered by a top solder mask layer and a top overlay. The bottom-most signal layer can be covered by a bottom solder mask layer and a bottom overlay.

In some embodiments, at least one of the dielectric layers can be a polyimide layer.

In some embodiments, the flexible printed circuit can comprise a first set of through-holes and a second set of through-holes. The first set of through-holes can be positioned distal to the second set of through-holes. At least one through-hole of the first set of through-holes can be electrically coupled to at least one through-hole of the second set of through-holes via the conductive traces of one of the signal layers.

In some embodiments, the first set of through-holes can comprise a first distal-most through-hole and a first proximal-most through-hole. The first distal-most through-hole can be electrically coupled to one of the through-holes of the second set of through-holes via a first conductive trace of one of the signal layers. The first proximal-most through-hole can be electrically coupled to another one of the through-holes of the second set of through-holes via a second conductive trace of another one of the signal layers.

In some embodiments, each of the first set of through-holes can have a hole diameter. In some embodiments, the hole diameter can be between 0.10 mm and 0.30 mm.

In some embodiments, at least one conductive wire can be coupled to one of the signal layers via one of the second set of through-holes.

In some embodiments, at least one conductive wire can comprise a wire distal end and a wire proximal end. The wire distal end can be coupled to the flexible printed circuit and the wire proximal end can be connected to a lead cable or directly to a telemetry unit. In some embodiments, the lead cable can be coupled to the telemetry unit.

In some embodiments, the paddle can comprise a conductive epoxy disposed on one side of the paddle. The first set of through-holes can be aligned with the conductive epoxy when the flexible printed circuit is coupled to the paddle.

In some embodiments, the conductive epoxy can be a conductive silver epoxy.

In some embodiments, each of the electrodes on the carrier body can be electrically coupled to one of the first set of through-holes via the conductive epoxy and conductive traces extending through the carrier body.

In some embodiments, the paddle can be coupled to the flexible printed circuit via an adhesive.

In some embodiments, the thickness of the flexible printed circuit can be between 0.20 mm and 0.25 mm.

In some embodiments, the thickness of the paddle can be between 0.10 mm and 0.30 mm.

In some embodiments, the flexible printed circuit can be a flexible printed circuit strip.

In some embodiments, the flexible printed circuit strip can have a strip length and a strip width. The strip length can be between about 15.0 mm to about 20.0 mm. The strip width can be between about 1.0 mm and 2.0 mm.

In some embodiments, the paddle can have a paddle length. The paddle length can be between about 8.0 mm and 10.0 mm.

In some embodiments, an electrical interface for an implantable electrode array is disclosed. The electrical interface can comprise a flexible printed circuit configured to be coupled to an implantable carrier. The flexible printed circuit can comprise one or more conductive wires coupled to the flexible printed circuit. The flexible printed circuit can comprise a plurality of layers stacked on top of one another. The flexible printed circuit can comprise a first set of through-holes and a second set of through-holes. The first set of through-holes can be positioned distal to the second set of through-holes. At least one through-hole of the first set of through-holes can be electrically coupled to at least one through-hole of the second set of through-holes via conductive traces disposed on one of the plurality of layers.

In some embodiments, the flexible printed circuit can comprise a plurality of signal layers separated by dielectric layers. At least one of the signal layers can comprise conductive traces.

In some embodiments, the signal layers can comprise a top-most signal layer and a bottom-most signal layer. The top-most signal layer can be covered by a top solder mask layer and a top overlay. The bottom-most signal layer can be covered by a bottom solder mask layer and a bottom overlay.

In some embodiments, at least one of the dielectric layers can be a polyimide layer.

In some embodiments, the first set of through-holes can comprise a first distal-most through-hole and a first proximal-most through-hole. The first distal-most through-hole can be electrically coupled to one of the through-holes of the second set of through-holes via a first conductive trace of one of the signal layers. The first proximal-most through-hole can be electrically coupled to another one of the through-holes of the second set of through-holes via a second conductive trace of another one of the signal layers.

In some embodiments, each of the first set of through-holes can have a hole diameter. In some embodiments, the hole diameter can be between 0.10 mm and 0.30 mm.

In some embodiments, at least one conductive wire can be coupled to one of the signal layers via one of the second set of through-holes.

In some embodiments, at least one conductive wire can comprise a wire distal end and a wire proximal end. The wire distal end can be coupled to the flexible printed circuit. The wire proximal end can be connected to a lead cable or directly to a telemetry unit.

In some embodiments, the lead cable can be coupled to the telemetry unit.

In some embodiments, the flexible printed circuit can be configured to be coupled to a part of the implantable electrode array via an adhesive and a conductive epoxy.

In some embodiments, the conductive epoxy can be a conductive silver epoxy.

In some embodiments, the implantable electrode array can comprise a plurality of electrodes. Each of the electrodes can be electrically coupled to one of the first set of through-holes via the conductive epoxy.

In some embodiments, the thickness of the flexible printed circuit can be between 0.20 mm and 0.25 mm.

In some embodiments, the flexible printed circuit can be a flexible printed circuit strip.

In some embodiments, the flexible printed circuit strip can have a strip length and a strip width. In some embodiments, the strip length can be between about 15.0 mm to about 20.0 mm. In some embodiments, the strip width can be between about 1.0 mm and 2.0 mm.

Also disclosed is an implantable electrode array. The implantable electrode array can comprise one or more shape-memory support structures and a carrier body encapsulating the one or more shape memory support structures.

The implantable electrode array can further comprise a plurality of conductive traces embedded within the carrier body and extending throughout portions of the carrier body. The implantable electrode array can also comprise a plurality of electrodes coupled to the carrier body. Each of the conductive traces can be coupled or connected to one of the electrodes. At least a part of each of the electrodes can be exposed by the carrier body 54 to allow the electrode to capture a signal such as a neural signal.

In some embodiments, the shape memory support structures can be made in part of a shape memory metal alloy. For example, the shape memory metal alloy can be a nickel-titanium alloy (e.g., Nitinol).

In some embodiments, the shape memory support structures can be made in part of a shape memory polymer.

The carrier body can be made in part of a liquid crystal polymer. The carrier body can be flexible and collapsible. In some embodiments, the carrier body can have or be defined by a digitate structure comprising a plurality of carrier fingers or leaflets.

At least one of the conductive traces can be made in part of gold or platinum. Moreover, at least one of the electrodes can be made in part of gold or platinum.

The one or more shape memory support structures can be configured to collapse when the implantable electrode array is in a delivery configuration. The one or more shape memory support structures can be configured to expand when the implantable electrode array is in a deployed configuration to improve the ability of the carrier body to maintain vessel wall apposition.

Also disclosed is a connector lead cable assembly. The connector lead cable assembly can comprise a first lead cable and a second lead cable. The first lead cable can comprise a first lead proximal end, a first lead distal end, and a first lead intermediate segment in between the first lead proximal end and the first lead distal end. The second lead cable can comprise a second lead proximal end, a second lead distal end, and a second lead intermediate segment in between the second lead proximal end and the second lead distal end.

The first lead cable can further comprise a plurality of first lead connectors disposed at the first lead proximal end for connecting to electrical interfaces within a first port of an implantable receiver telemetry unit (IRTU). The second lead cable can further comprise a plurality of second lead connectors disposed at the second lead proximal end for connecting to electrical interfaces within a second port of the IRTU.

The first lead distal end and the second lead distal end can be configured to be coupled to an implantable electrode array.

The first lead intermediate segment can be configured to axially stack together with the second lead intermediate segment such that the first lead intermediate segment stacked together with the second lead intermediate segment can fit within a single small diameter delivery catheter.

The first lead cable can comprise a first cable body and a plurality of first conductive wires extending through the first cable body. The second lead cable can comprise a second cable body and a plurality of second conductive wires extending through the second cable body.

In some embodiments, the first cable body and the second cable body can be made in part of a liquid crystal polymer.

In some embodiments, the second lead intermediate segment can have a substantially U-shaped cross-section. The first lead intermediate segment can have a substantially circular cross-section. The first lead intermediate segment can be sized to fit within a substantially U-shaped trough of the second lead intermediate segment when the first lead intermediate segment is axially stacked together with the second lead intermediate segment.

In some embodiments, the first lead cable can comprise between three and twelve first lead connectors. The second lead cable can comprise between three and twelve second lead connectors.

### BRIEF DESCRIPTION OF THE DRAWINGS

FIG. 1A illustrates a system for implanting electrodes in the form of a tubular member.

FIG. 1B illustrates a close-up view of part of the tubular member of FIG. 1A.

FIG. 1C illustrates a cross-sectional view of the tubular member of FIG. 1A taken along cross-section A-A.

FIG. 2A illustrates another variation of a system for implanting electrodes in the form of a tubular member.

FIG. 2B illustrates a cross-sectional view of the tubular member of FIG. 2A taken along cross-section B-B.

FIGS. 2C and 2D illustrate cross-sectional views of different variations of the tubular member of FIG. 2A.

FIG. 3 illustrates a multi-lumen device combining multiple tubular members and an unraveled electrode array to access multiple vessels.

FIG. 4 illustrates a method for positioning the tubular member comprising electrodes against a vessel wall.

FIG. 5A illustrates a variation of a tubular member with electrodes configured to penetrate through a vessel wall (e.g., a cerebral vessel wall).

FIG. 5B illustrates a top view of an electrode base of an electrode comprising electrode spikes or pointed ends.

FIGS. 5C and 5D illustrate side views of different variations of electrodes with electrode spikes or pointed ends.

FIG. 5E illustrates a variation of an electrode comprising electrode spikes or pointed ends that can be used to carry and deliver therapeutic agents (e.g., pharmaceuticals or medicaments).

FIGS. 6A to 6G illustrate variations of an implantable electrode array comprising a lead device carrying one or more electrodes.

FIG. 7A illustrates a system for implanting a plurality of electrodes within a cerebral vessel of a subject.

FIG. 7B illustrates an inner tubular member and an outer tubular member of the system in a disassembled configuration.

FIG. 8A illustrates one embodiment of a stent comprising electrodes for implantation in a cerebral vessel.

FIG. 8B illustrates various layers of one embodiment of part of a paddle of the stent of FIG. 8A.

FIG. 8C illustrates another embodiment of part of the paddle of the stent.

FIG. 8D illustrates one embodiment of the paddle comprising two conductive tracks that connect to each of a plurality of electrodes.

FIG. 8E illustrates two cross-sections of part of a strut of the stent.

FIG. 9 illustrates one embodiment of an implantable electrode array for recording neural activity and/or stimulating neural tissue within the brain of a subject.

FIG. 10 illustrates a side cross-sectional view of part of a flexible printed circuit of an electrical interface of the implantable electrode array.

FIG. 11 illustrates a top view of a plurality of signal layers of the flexible printed circuit.

FIG. 12 illustrates the flexible printed circuit implemented as a substantially elongated rectangular flexible printed circuit strip.

FIG. 13 illustrates the conductive wires directly coupled to the flexible printed circuit coupled to a paddle of an implantable electrode array.

FIG. 14A illustrates one embodiment of an electrode of an implantable electrode array.

FIG. 14B illustrates that the electrode of FIG. 14A can be placed or positioned within an electrode window arranged between adjacent struts of the implantable electrode array.

FIG. 15 is a close-up view of an electrode mounting site of one of the stents or stent electrode arrays disclosed herein.

FIG. 16A illustrates one embodiment of an unraveled electrode array.

FIG. 16B illustrates a close-up view of part of the unraveled electrode array.

FIG. 17A illustrates one embodiment of a stent electrode array comprising a substantially tubular stent body comprising struts forming a plurality of undulating rings and electrodes serving as ring connectors.

FIG. 17B illustrates another embodiment of a stent electrode array comprising a substantially tubular stent body comprising struts forming a plurality of deformed hoops and electrodes serving as hoop connectors.

FIG. 18A illustrates another embodiment of a stent electrode array comprising a substantially tubular stent body comprising struts forming a plurality of piriform structures and electrodes serving as distal ends or tips of the piriform structures.

FIG. 18B illustrates another embodiment of a stent electrode array comprising a substantially tubular stent body comprising struts forming a plurality of pointed oval structures and electrodes serving as distal ends or tips of the pointed oval structures.

FIG. 19A illustrates another embodiment of a stent electrode array comprising a stent body comprising struts forming alternating leaf-like wire structures and electrodes coupled to the leaf-like wire structures.

FIG. 19B illustrates another embodiment of a stent electrode array comprising a stent body comprising struts forming collapsed hoops and electrodes coupled to the collapsed hoops.

FIG. 20A illustrates another embodiment of a stent electrode array comprising a substantially tubular stent body comprising struts forming a plurality of limoniform structures and electrodes serving as distal ends or tips of the limoniform structures.

FIG. 20B illustrates another embodiment of a stent electrode array comprising a substantially tubular stent body comprising struts forming a plurality of deformed limoniform structures and electrodes serving as distal ends or tips of the deformed limoniform structures.

FIG. 21A illustrates a top plan view of one embodiment of an implantable electrode array in a flattened configuration.

FIG. 21B illustrates an example cross-sectional view of the implantable electrode array of FIG. 21A taken along cross-section C-C.

FIG. 22A illustrates one embodiment of a flexible wire comprising electrodes coupled along a length of the flexible wire for implantation into a cerebral vessel.

FIG. 22B illustrates one embodiment of a coiled wire comprising electrodes coupled along a length of the coiled wire for implantation into a cerebral vessel.

FIG. 23A illustrates one embodiment of a bendable wire comprising electrodes coupled along a length of the bendable wire for implantation into a cerebral vessel.

FIG. 23B illustrates one embodiment of a coiled wire comprising electrodes coupled along a length of the coiled wire for implantation into a cerebral vessel.

FIG. 24A illustrates one embodiment of a connector lead cable assembly in the process of connecting to one embodiment of an implantable receiver telemetry unit.

FIG. 24B illustrates a first lead cable of the connector lead cable assembly axially stacked together with a second lead cable of the connector lead cable assembly.

FIG. 24C illustrates a cross-sectional view of the first lead cable axially stacked together with the second lead cable of FIG. 24B taken along cross-section D-D of FIG. 24B.

FIG. 24D illustrates a plurality of conductive wires extending from cable bodies of the connector lead cable assembly coupled to a flexible printed circuit.

FIG. 25 illustrates certain cerebral veins and sinuses of a subject that can serve as possible implantation sites for the various devices and systems disclosed herein.

### DETAILED DESCRIPTION

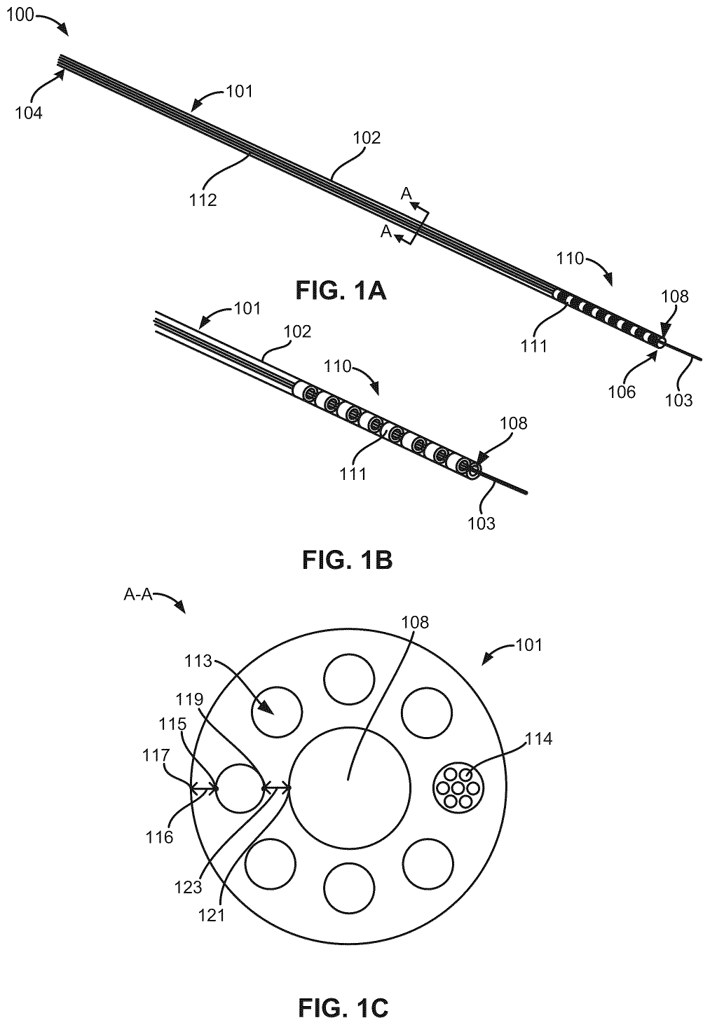

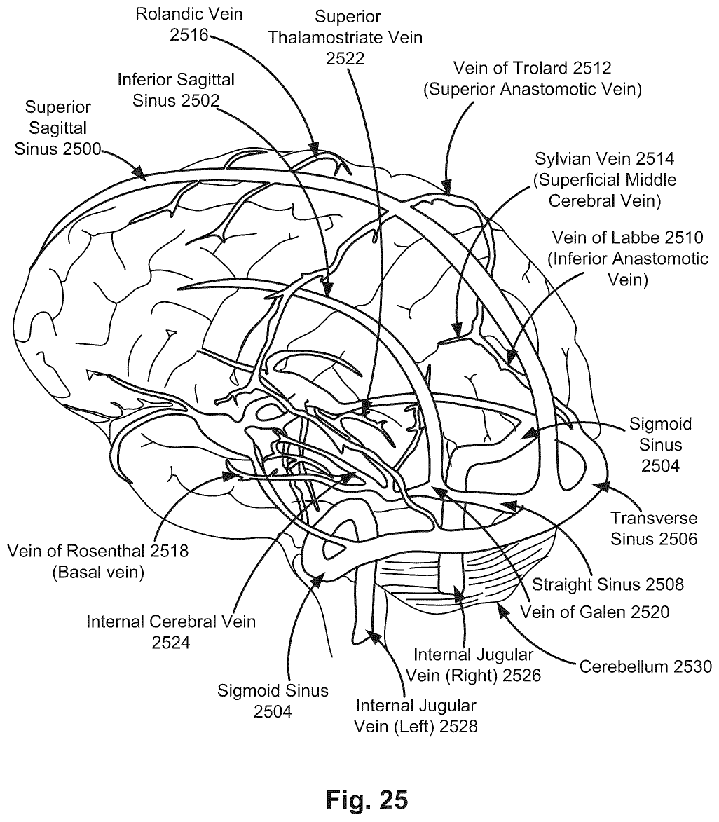

FIG. 1A illustrates a system 100 for implanting electrodes 111 within a cerebral vessel (see FIG. 25) of a subject. The system 100 can comprise a tubular member 101 for placement over a microwire 103. The tubular member 101 can comprise a tubular body 102, a proximal end 104, a distal end 106, and a hollow central lumen 108 extending therebetween. The tubular member 101 can be configured to be threaded over the microwire 103 via the central lumen 108. The microwire 103 can be delivered to a desired location/target site prior to advancement of the tubular member 101. The microwire 103 can be removed after the tubular member 101 is deployed at the target site within the cerebral vessel.

The tubular member 101 can comprise an electrode array 110 coupled to the tubular body 102. The electrode array 110 can comprise one or more flexible ring electrodes 111 coupled to the tubular body 102 along at least part of the length of the tubular body 102. The electrode array 110 can be considered the “active” portion of the tubular member 101.

The tubular body 102 can be sized to be delivered through one or more cerebral vessels and implanted at an implantation site within a cerebral vessel. The electrodes 111 of the electrode array 110 can be used to record neural activity or stimulate neighboring neural tissue. The electrodes 111 of the electrode array 110 can be electrically coupled to a telemetry unit that is implanted within the subject or an extracorporeal telemetry unit that is located outside the body of the subject.

The tubular body 102 can have a body length. In some embodiments, the body length can be between about 80 mm and 120 mm (e.g., about 100 mm). The tubular body 102 can comprise an outer diameter equivalent to about 2.3 French and hence suitable for delivery into smaller branching cerebral vessels.

In some embodiments, the tubular body 102 can comprise an atraumatic tip. The atraumatic tip can be soft and/or blunted so as to not pierce, dissect, or damage tissue during delivery and deployment.

FIG. 1B illustrates a close-up view of a distal segment of the system 100. As shown in FIG. 1B, a plurality of spaced-apart flexible ring electrodes 111 can be affixed, secured, or otherwise coupled around an external body wall of the tubular body 102. The ring electrodes 111 can be spaced apart along at least part of a distal segment of the tubular body 102. The electrode array 110 can also have a multitude of unidirectional electrodes coupled to the tubular body 102 at different orientations.

In some embodiments, the electrodes 111 can be spaced apart by about 2.0 mm intervals. In other embodiments, the electrodes 111 can be spaced apart by about 1.0 mm, 1.5 mm, 2.5 mm, 3.5 mm, 4.0 mm, 4.5 mm, or 5.0 mm intervals.

FIG. 1C illustrates a cross-sectional view of the tubular member 101 of FIG. 1A taken along cross-section A-A. As shown in FIG. 1C, the tubular member 101 can comprise one or more peripheral lumens 113 surrounding the central lumen 108. The peripheral lumens 113 and the central lumen 108 can extend through the tubular body 102.

In some embodiments, the tubular member 101 can comprise between four and eight peripheral lumens 113. For example, the tubular member 101 can comprise seven peripheral lumens 113.

The peripheral lumens 113 can radially surround the central lumen 108.

Each of the peripheral lumens 113 can have a radially outermost point 115 furthest from a peripheral lumen center. The radially outermost point 115 can be separated from an exterior surface 117 of the tubular body 102 by a lumen-wall separation distance 116. In some embodiments, the lumen-wall separation distance 116 can be at least 25 μm.

Each of the peripheral lumens 113 can also have a radially innermost point 119. The radially innermost point 119 furthest from a peripheral lumen center can be separated from a radially outermost point 121 of the central lumen 108 further from a central lumen center by a lumen-lumen separation distance 123. In some embodiments, the lumen-lumen separation distance 123 can be at least 25 μm.

The peripheral lumens 113 can be configured to allow a bundle of conductive wires 114 to pass therethrough. The conductive wires 114 can be covered by an insulator (e.g., polyurethane) or an insulating coating or layer.

Each of the peripheral lumens 113 can have an inner diameter between about 100 μm and about 200 μm. For example, each of the peripheral lumens 113 can have an inner diameter of about 150 μm.

The tubular member 101 can have an outer diameter between about 600 μm and about 800 μm. For example, the tubular member 101 can have an outer diameter of about 700 μm.

The central lumen 108 can have an outer diameter between about 300 μm and about 500 μm. For example, the central lumen 108 can have an outer diameter of about 400 μm.

The conductive wires 114 can each have a diameter of between about 40 μm to 60 μm (e.g., 50 μm).

In some embodiments, the microwire 103 can have a wire diameter of between about 300 μm and about 400 μm (e.g., about 356 μm). In other embodiments, the microwire 103 can have a wire diameter of less than about 300 μm. The total length of the tubular body 102 can be between about 80 mm and about 120 mm (e.g., about 100 mm).

Each of the electrodes 111 can be spaced apart from its nearest neighboring electrode 111 by about 2.0 mm to about 4.0 mm (e.g., about 2.0 mm to about 3.0 mm).

The peripheral lumens 113 can each comprise a bundle of seven conductive wires 114 extending therethrough. In the embodiment shown in FIG. 1C, the bundle of conductive wires 114 can have a total bundle diameter of slightly less than about 150 μm. The bundle can be electrically insulated. In some embodiments, the bundle of wires 114 can comprise between two wires 114 and seven wires 114. A conductive portion of each of the wires 114 can be connected to each one of the electrodes 111.

In one embodiment, the tubular member 101 can comprise 56 electrodes 111. In some embodiments, the tubular member 101 can comprise between 32 electrodes 111 and 56 electrodes 111.

In some embodiments, each of the wires 114 can lead into one electrode 111. For example, a hole can be made in the peripheral lumen 113 where a wire 114 is exposed. The wire 114 can be electrically connected to the electrode 111 accordingly.

The holes in the peripheral lumen 113 can be positioned at the end of the tubular body 102 or along its length. For example, the holes can be staggered along the length of the tubular body 102 such that the connections to the electrodes 111 are staggered throughout.

In some embodiments, the bundle can be fed through one hole in the peripheral lumen 113. In some embodiments, a single wire 114 from the bundle can be exposed and electrically connected to an electrode 111. In some embodiments, each wire bundle can either be electrically insulated or put into a sleeve to maintain the bundle of wires together.

Referring back to FIGS. 1A and 1B, in some embodiments, the electrodes 111 can be cylindrical or ring electrodes 111. The cylindrical or ring electrodes 111 can be omnidirectional electrodes that completely circumferentially surround the tubular body 102.

In other embodiments, the electrodes 111 can be cuff-shaped, partially ring-shaped (e.g., C-shaped), or semi-cylindrical electrodes 111. The semi-cylindrical electrodes and the partially ring-shaped electrodes can be made by coating half of a cylinder or a ring. Alternatively, the semi-cylindrical electrodes and the partially ring-shaped electrodes can be made by laser cutting half of a cylinder electrode or ring electrode.

In some embodiments, the cuff electrodes can partially surround the tubular body 102 such that the electrodes 111 do not completely surround the tubular body 102. In addition, at least some of the cuff electrodes can be circumferentially misaligned with respect to one another along part of the length of the tubular body 102.

The electrodes 111 can be semicircular electrodes such that each of the semicircular electrodes surround half of a circumference of the tubular body 102. In some embodiments, the semicircular electrodes can be circumferentially offset with respect to one another along part of the length of the tubular body 102.

In some embodiments, the electrodes 111 can be positioned in a helical pattern along the length of the tubular body 102. A pitch of the helical pattern can be between about 2.0 mm and about 4.0 mm.

In some embodiments, the electrodes 111 of the electrode array 110 can be made in part of platinum, platinum black, another noble metal, or alloys or composites thereof.

In certain embodiments, the electrodes 111 of the electrode array 110 can be made of gold, iridium, palladium, a gold-palladium-rhodium alloy, rhodium, or a combination thereof.

In some embodiments, the electrodes 111 can be made of a metallic composite with a high charge injection capacity (e.g., a platinum-iridium alloy or composite).

One technical problem faced by the applicant is how to design a neural interface to be delivered to or through small cerebral vessels which are limited by the vessel diameter and the thinness of the vessel walls. One technical solution discovered and developed by the applicant is to use a tubular member designed to be delivered over the top of a deployed microwire eliminating the need for a large catheter to deliver the tubular member. Accordingly, the largest device to be introduced can be the tubular member itself and not a delivery catheter.

Another technical problem faced by the applicant is how to physically add more electrodes to a device used to stimulate neural targets and record neural activity in smaller cerebral vessels in particular. One technical solution discovered and developed by the applicant is to use a multi-lumen tubular member that does not need to be collapsed and expanded for delivery, allowing for more physical space for electrodes to be coupled thereto. As a result, in one example embodiment, a 256-channel neural recording and stimulating tubular member can be provided that can perform bi-directional recording and stimulation of neural targets.

The number of electrodes 111 utilized can be a function of the thickness of each of the individual wires 114, the thickness of the insulation of the wires 114, a lumen thickness of the tubular member 101, and a diameter of the tubular member 101. In some embodiments, more electrodes 111 can be included by reducing the diameter of the central lumen 108 or the lumen-wall separation distance 116.

The insulation for each of the wires 114 can have a thickness between about 6 μm to 8 μm (e.g., 7 μm) for a 50 μm wire. The insulation thickness can change depending on the wire and insulation materials and the dielectric properties that are required.

In some embodiments, the system 100 can comprise multiple tubular members 101 that can all be implanted within the subject. The system 100 can thus access the brain from multiple locations simultaneously (e.g., the Rolandic veins, the vein of Labbe, the vein of Trolard, the superior sagittal sinus, and the middle meningeal artery, etc.).

To develop the electrode array 110 disclosed herein, each of the proximal ends containing 50 or more electrodes can be laid down on top of a base plate. The base plate can have electrical contacts that align magnetically with the electrical contacts of the proximal ends. The magnetic alignment can utilize magnets as the proximal end contacts themselves, or with magnetic tags placed along the length of the proximal end in respective positions to those on the base plate. Further, the electrical contacts can be raised such that they assist with contacting the proximal ends. When the proximal ends are placed on the baseplate, the top plate can be placed over the top. The top plate can have a silicone gasket running around the entire length of the electrode array to prevent water ingress.

Further, silicone components or other insulating material can be provided between each contact location to prevent water-washed ionic shorting between individual contacts. As the top plate is positioned over the base plate, the curved base plate contacts become slightly depressed, enhancing contact. The base plate can then be screwed shut.

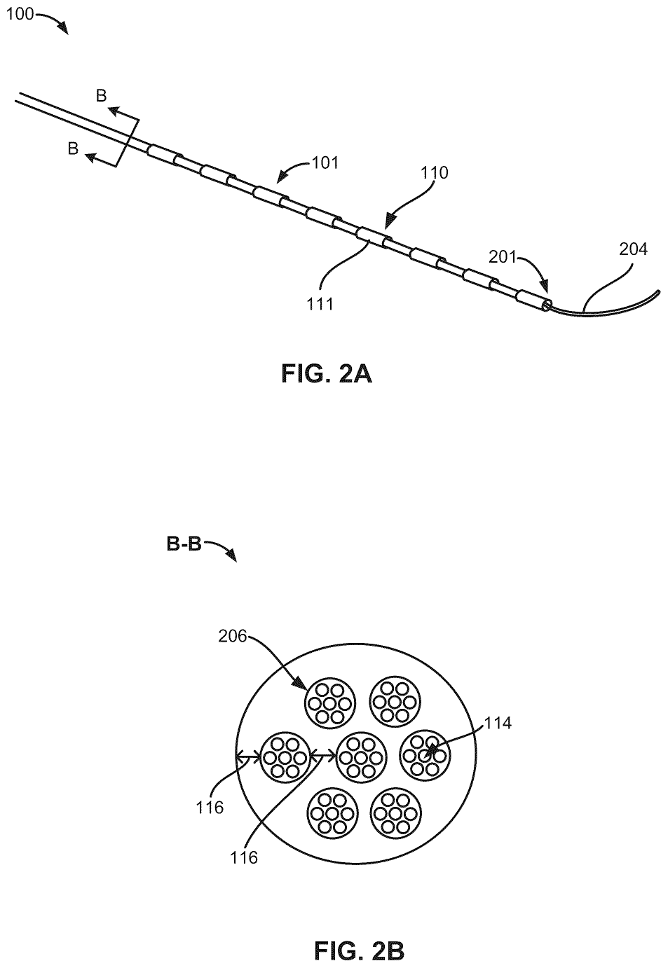

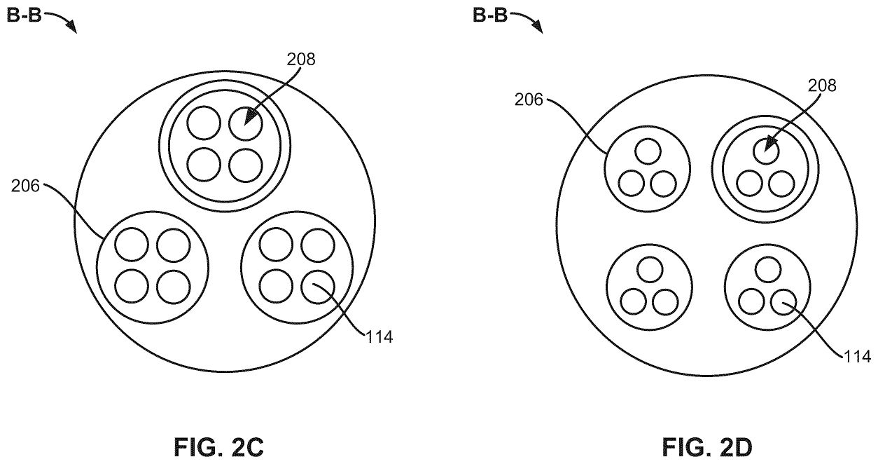

FIG. 2A illustrates another variation of a system 100 for implanting electrodes 111 within a cerebral vessel (see FIG. 25) of a subject. The system 100 can comprise another variation of the tubular member 101 with multiple lumens 206 but without accommodation for a microwire to extend through the tubular member 101 (i.e., with a dedicated lumen for the microwire).

In this embodiment, the tubular member 101 itself can be pushed into small cerebral vessels. The tubular member 101 can further comprise an angled soft tip 204 at its distal end 201 to aid the tubular member 101 in navigating through tortuous bends of small cerebral vessels. The tubular member 101 can be specifically designed to access vessels less than 1.0 mm in diameter.

FIG. 2B illustrates a cross-sectional view of the tubular member of FIG. 2A taken along cross-section B-B. Each of the lumens 206 of the tubular member 101 of FIG. 2A can have an inner diameter of about 150 μm. The lumens 206 can be separated from one another by a lumen-wall separation distance 116 of about 25 μm. The tubular member 101 can have an outer diameter of about 550 μm. The tubular member 101 can comprise a plurality of conductive wires 114 extending through each of the lumens 206. The conductive wires 114 can each have a diameter of about 50 μm.

In one embodiment, the tubular member 101 of FIG. 2A can comprise 49 electrodes and seven bundles of seven conductive wires 114 for connecting to such electrodes.

FIGS. 2C and 2D are cross-sectional views of different variations of the tubular member 101 of FIG. 2A taken along cross-section B-B. For example, in the variation shown in FIG. 2C, twelve wire bundles 208 can be provided in three groups of four, resulting in an outer diameter of 756 μm for the tubular member 101.

Also, for example, in the variation shown in FIG. 2D, twelve wire bundles 208 can be provided in four groups of three, resulting in an outer diameter of 786 μm for the tubular member 101.

Each wire bundle 208 can comprise a plurality of conductive wires 114 with each conductive wire 114 configured to be coupled to an individual electrode 111. The conductive wires 114 can each be 40 μm in diameter.

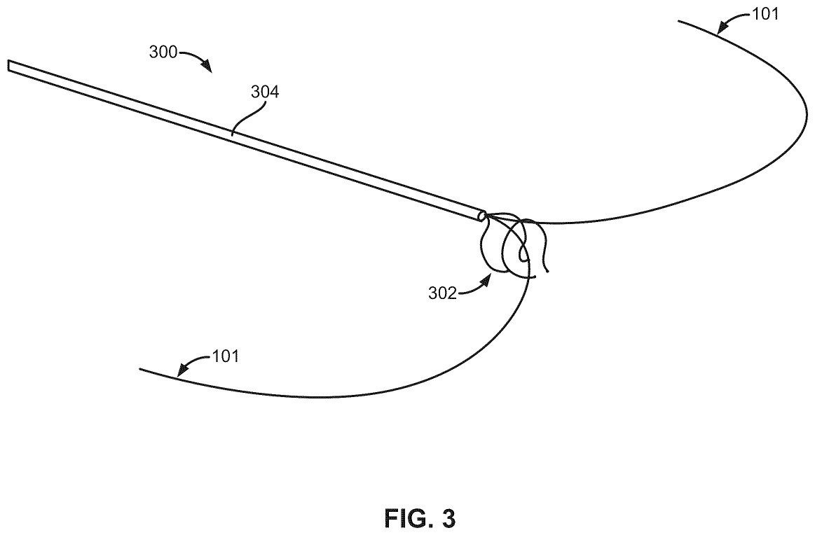

FIG. 3 illustrates a multi-lumen device 300 comprising multiple tubular members 101 (for example, any of the tubular members 101 shown in FIGS. 1A and 2A) and an unraveled electrode array 302. The multi-lumen device 300 can access multiple cerebral vessels.

In some embodiments, the multi-lumen device 300 can comprise a central tube 304 or delivery catheter that can carry the tubular members 101 and the unraveled electrode array 302.

In some embodiments, the multiple tubular members 101 can share a single transmission lead or each of the tubular members 101 can be connected to its own transmission lead. Each of the tubular member 101 can comprise an electrode array 110 (similar to the electrode arrays 110 shown in FIGS. 1A and 2A) comprising a plurality of electrodes 111.

In certain embodiments, at least part of the central tube 304 can be delivered to the superior sagittal sinus and the various tubular members 101 and the unraveled electrode array 302 can access the smaller branching vessels connected to the superior sagittal sinus.

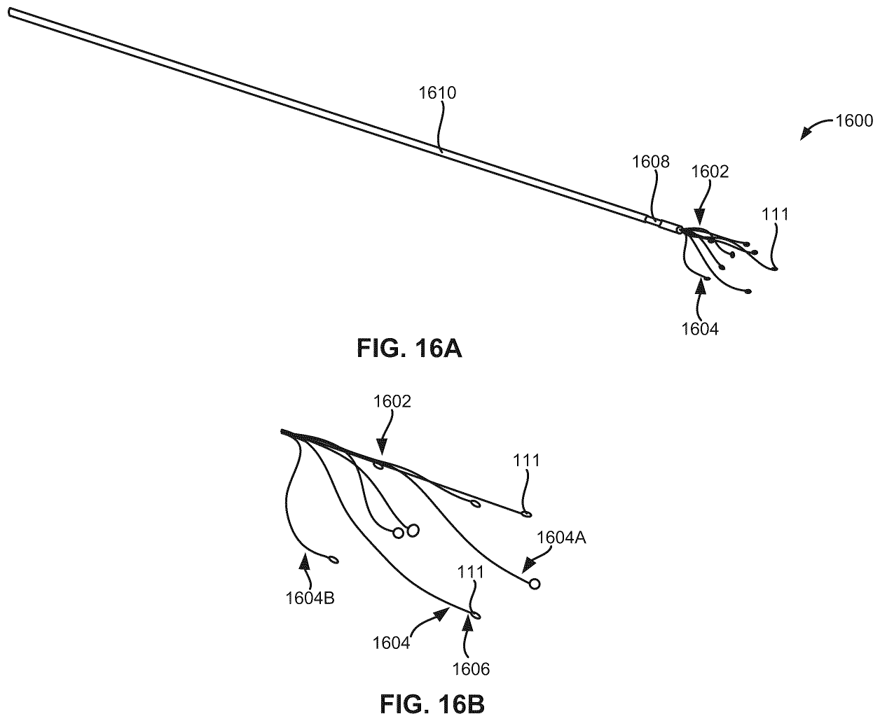

In some embodiments, the unraveled electrode array 302 shown in FIG. 3 can be the same unraveled array 1600 shown in FIGS. 16A and 16B.

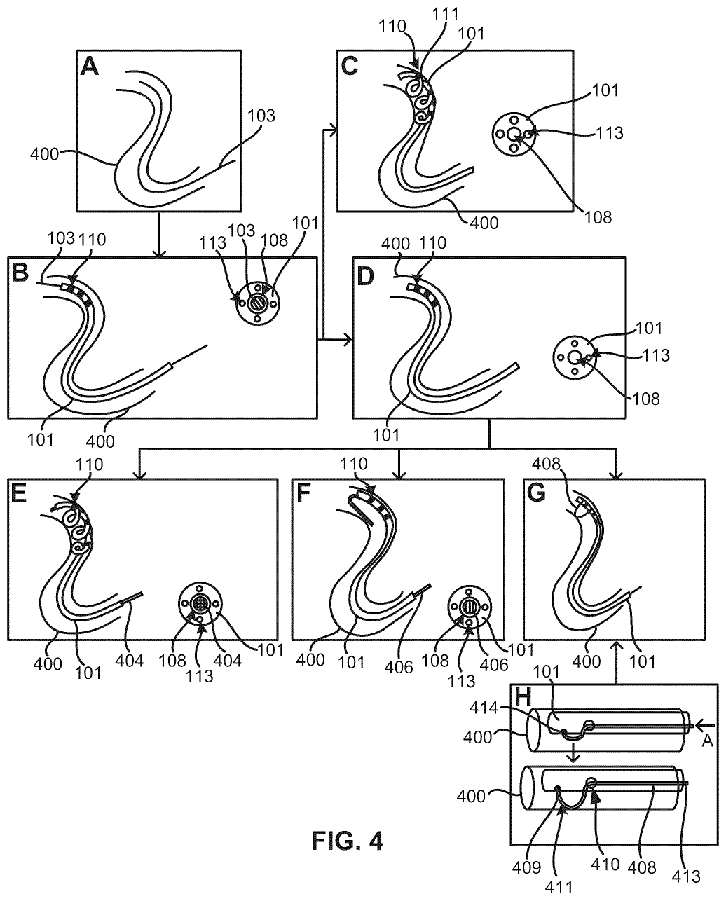

FIG. 4 illustrates a method for delivering and deploying several variations of a tubular member 101 within a cerebral vessel 400. The method can begin with delivering a microwire 103 into the cerebral vessel 400 in Step A. For example, the microwire 103 can be delivered to a target site within the cerebral vessel 400. The target site can be an area within the brain or a region of the brain to be stimulated or sensed (via neural signal recordings). The cerebral vessel 400 can be any of the cerebral veins or sinuses shown in FIG. 25.

A tubular member 101 can then be advanced over the microwire 103 via the central lumen 108 to the target site in Step B. The tubular member 101 can have a plurality of peripheral lumens 113 configured to carry conductive wires 114 (see, e.g., FIGS. 1C and 2B-2D). Each of the conductive wires 114 can be electrically coupled to an electrode 111 of the electrode array 110. In some embodiments, Step B can be the final step if the electrode array 110 of the tubular member 101 is placed against the vessel wall of the cerebral vessel 400 and in good apposition with the vessel wall adjacent to the desired target site. The microwire 103 can be removed when the tubular member 101 is at the target site.

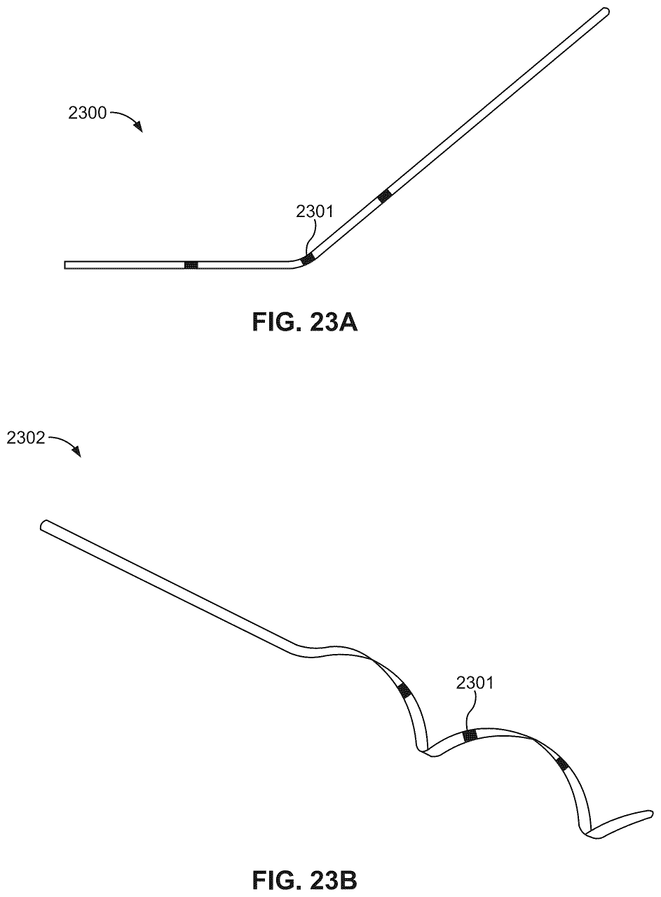

In certain embodiments, such as what is shown in Step C, the tubular member 101 can self coil or recover a pre-set coiled configuration when the microwire 103 is removed. In these embodiments, the tubular member 101 can be the coiled wire 2302 shown in FIG. 23B.

In other embodiments, the tubular member 101 does not self coil or recover a pre-set coiled configuration when the microwire 103 is removed (as shown in Step D). In these embodiments, one option is to insert a pre-shaped helical wire 404 or coiled wire through the central lumen 108 (which is now empty since the microwire 103 has been removed) in Step E. The pre-shaped helical wire 404 or coiled wire can twist the tubular member 101 into a helical or coiled configuration in response. In the helical or coiled configuration, the electrode array 110 carried by the tubular member 101 can be pushed against the vessel wall of the cerebral vessel 400 and in good apposition with the vessel wall.

In some embodiments, after removal of the microwire 103, a curved microwire 406 can be inserted through the central lumen 108 of the tubular member 101 as shown in Step F. The curved microwire 406 can place tension on the tubular member 101 and can force the electrode array 110 carried by the tubular member 101 to be pushed against the vessel wall in good apposition with the vessel wall of the cerebral vessel 400.

In other embodiments, after removal of the microwire 103, an anchoring wire 408 can be secured, adhered, or otherwise attached to the tubular member 101 as shown in Step G.

The anchoring wire 408 can comprise a wire distal end 409, an anchoring segment 411, and a proximal wire segment 413 proximal to the anchoring segment 411. The anchoring wire 408 can be sized to extend through one of the peripheral lumens 113 or another lumen extending through the tubular member 101.

The distal end of the anchoring wire 408 can be coupled to a distal point 414 along a tubular body wall of the tubular member 101. At least part of the anchoring segment 411 of the anchoring wire 408 can be configured to extend through an opening 410 defined along the tubular body wall of the tubular member 101. The opening 410 can be positioned proximal to the distal point 414. At least part of the anchoring segment 411 can be configured to form a looped or curved segment outside of the tubular body when part of the proximal wire segment 413 is advanced distally. The looped anchoring segment 411 can be configured to secure the tubular member 101 within the cerebral vessel.

Forward pressure (arrow A) on the anchoring wire 408 can push the anchoring wire 408 out through the opening 410 (as shown in Step H) to force the anchoring wire 408 into an anchored state and push the electrodes 111 of the electrode array 110 (carried by the tubular member 101) against the vessel wall of the cerebral vessel. Although FIG. 4 illustrates one anchoring wire 408, it is contemplated by this disclosure that multiple anchoring wires 408 can be used to anchor or secure the tubular member 101 within the cerebral vessel.

One technical problem faced by the applicants is how to secure or anchor an electrode array 110 carried by a wire-type carrier against a vessel wall while preventing migration of the electrode array 110. Migration can lead to vascular damage or issues with signal recording or inadequate stimulation when the electrode array 110 migrates or becomes unsecured. One technical solution discovered and developed by the applicants is to secure an electrode array 110 against the vessel wall of a cerebral vessel using the methods disclosed herein.

For example, anchoring can be achieved via a looped or curved anchoring segment 411 (see Step H), a self-expanding or self-coiling tubular member 101 (see Step C), or a forced coiling of the tubular member (see Step E).

In some embodiments, the microwire 103 can comprise an anchoring barb defined or otherwise coupled to a distal end of the microwire 103. The anchoring barb can allow the microwire 103 to create a puncture in the vessel wall to allow the microwire 103 to extend through the vessel wall. The microwire 103 can then direct the electrode array 110 closer to the target site such that the electrode array 110 is positioned at or in close proximity to the intracorporeal target within the cerebral vessel.

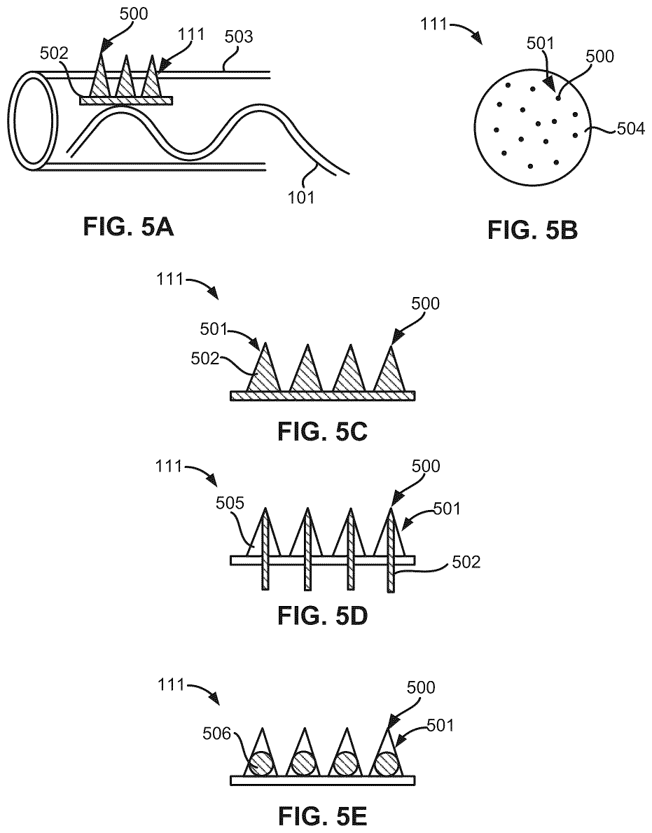

FIG. 5A illustrates a variation of the tubular member 101 with electrodes 111 configured to penetrate through a vessel wall 503 (e.g., a cerebral vessel wall). The electrodes 111 can comprise a plurality of electrode bodies 501 that each converges at a sharp point 500. In some embodiments, the sharp points 500 can be made of electrically conductive material 502 throughout.

The plurality of electrode bodies 501 can comprise at least a first electrode body and a second electrode body. The plurality of electrode bodies 501 can extend from an electrode base 504. The sharp points 500 can penetrate through the vessel wall 503 (e.g., the cerebral vessel wall) and into cortical or neural tissue without adversely affecting the vessel wall 503 or damaging the electrodes 111 when the electrode 111 is pressed against the vessel wall 503.

The first electrode body can have a first electrode body height. The second electrode body can have a second electrode body height. The first electrode body height can be greater than the second electrode body height.

In some embodiments, the first electrode and the second electrode can be connected to the conductive portion of the same wire 114 or, respectively, a first wire and second wire of a plurality of conductive wires. The first wire and the second wire can extend through the same peripheral lumen 113 or different peripheral lumens 113.

Once the implantable electrode array disclosed herein is deployed within a vessel wall 503, self-expanding forces or balloon expansion forces can push the electrodes 111 through the vessel wall 503. This can place the electrodes 111 closer to certain neural targets, resulting in an increased signal-to-noise ratio, reduced noise artifacts, and reduced stimulation charge delivery requirements.

FIG. 5B illustrates a top view of an electrode base 504 of one embodiment of the electrode 111. The electrode base 504 can comprise a plurality of electrode bodies 501 with sharp points 500 scattered throughout the area of the electrode base 504. The electrode bodies 501 can be less than about 0.5 mm wide and greater than about 0.5 mm high. The electrode base 504 can be substantially flat or curved. The electrode bodies 501 on the electrode base 504 can vary in height.

FIGS. 5C and 5D illustrate side views of variations of electrodes 111 with different types of sharp points 500 or converging spikes. For example, FIG. 5C illustrates electrodes 111 with electrode bodies 501 made of a single electrically conductive material 502.

FIG. 5D illustrates one embodiment of the electrodes 111 with electrode bodies 501 made of an electrically conductive material 502 and an insulating medium 505. For example, electrode bodies 501 can be made mostly of the insulating medium 505 except for a plurality of elongated spikes extending through the insulating medium 505 where each of the elongated spikes is made of an electrically conductive material 502. The distal ends of the elongated spikes serve as the sharp points 500 or converging spikes of the electrodes 111. Each of the sharp points 500 or converging spikes can be surrounded by an insulating medium 505 such that neighboring micro-electrode sharp points 500 do not connect or contact one another.

FIG. 5E illustrates electrode bodies 501 that can be used as carriers for pharmaceuticals 506 or other types of therapeutic agents or medicaments (e.g., therapeutic drugs, including but not limited to materials with anti-thrombogenic properties). The pharmaceuticals 506 or other types of therapeutics can be released when the electrodes 111 are implanted within the subject. Release of such pharmaceuticals 506 can be triggered via active opening of the electrode bodies 501 via an electrical current or electrical stimulation applied to the electrodes 111 or deliberately breaking open the electrode bodies 501 by mechanical force. For example, the electrode body 501 can be configured to release the pharmaceuticals 506 when the electrodes 111 are pressed against the vessel wall. In some embodiments, release of the pharmaceuticals 506 can be passive through diffusion.

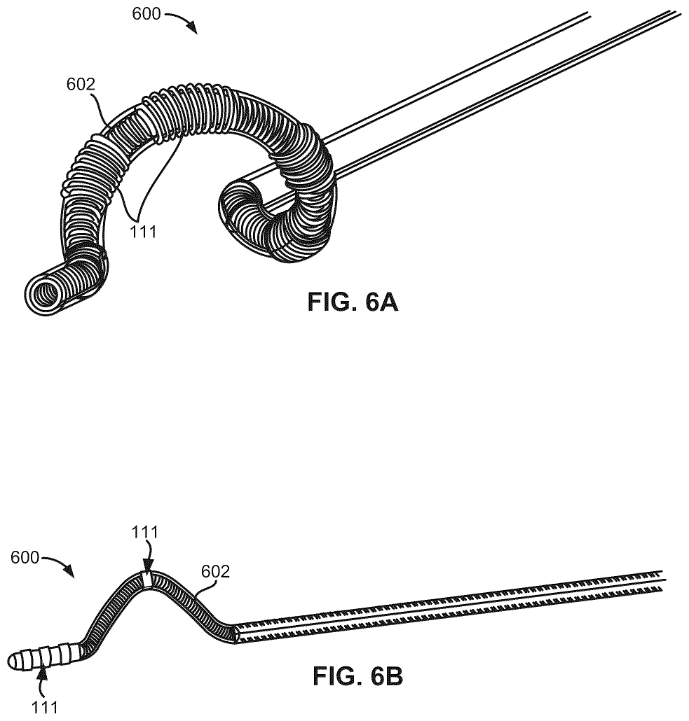

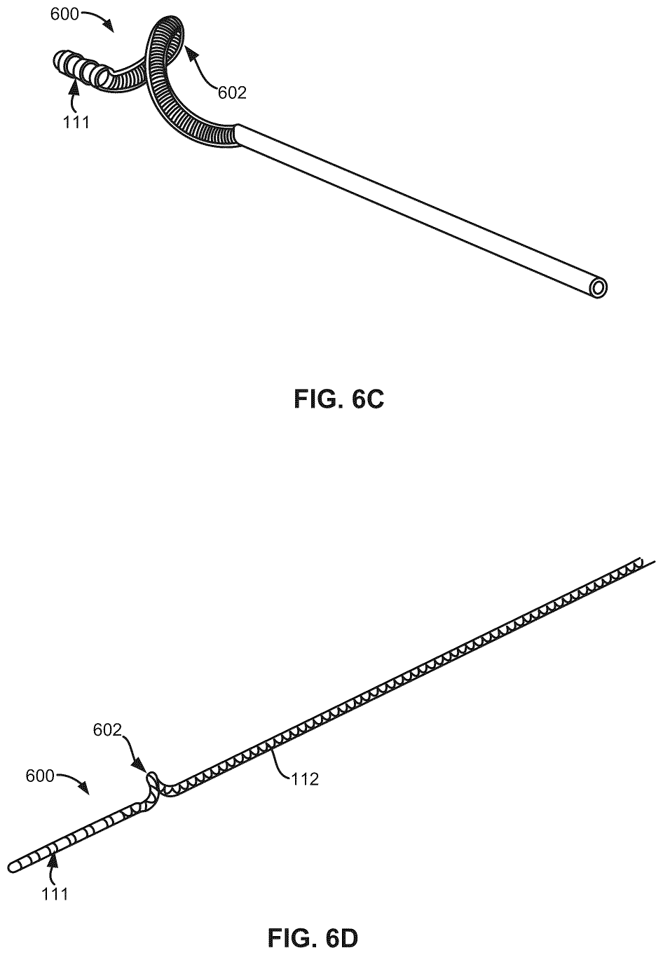

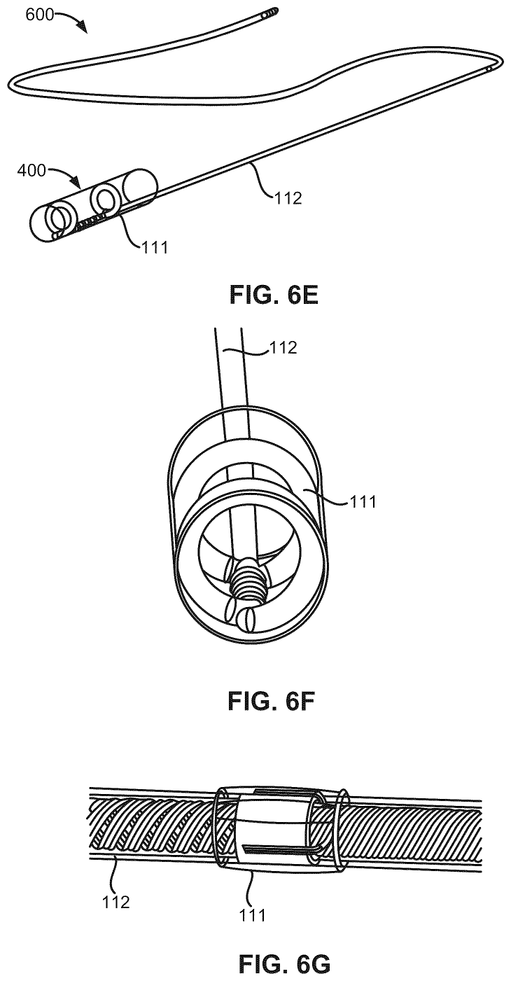

FIGS. 6A to 6G illustrate variations of a lead device 600 for accessing small neurovascular cerebral vessels. The lead device 600 can comprise a plurality of flexible regions that allow the lead device 600 to be easily delivered and deployed. In some embodiments, the lead device 600 can be made of a polymeric material. In other embodiments, the lead device 600 can be made of a metal hypotube.

The lead device 600 can have a curved section 602 positioned along the lead device 600. The curved section 602 can be in the form of a helix, a sine curve, curved bend, or a combination thereof.

The curved section 602 of the lead device 600 can be pre-set at a predetermined dimension and a predetermined shape. The curved section 602 can comprise one or more electrodes 111.

The curved section 602 can straighten out via material stress during advancement of the lead device 600 via a delivery catheter and over a guidewire to a target location in a cerebral vessel.

This lead device 600 can have one or more predefined or predetermined curves in it to enable it to provide force against the vessel wall to prevent migration and anchor the electrodes 111 in place. The lead device 600 can be straight during insertion and can form a curved or bent configuration when the delivery catheter housing the lead device 600 during the delivery/access phase is removed. When in the curved or bent configuration, the electrodes 111 of the lead device 600 can be forcibly pushed and/or secured against the vessel wall.

In some embodiments, the electrodes 111 can be cylindrical or ring electrodes (e.g., omnidirectional electrodes), semi-cylindrical electrodes or partially ring-shaped electrodes (e.g., directional electrodes), or electrodes made from strands of coiled metal.

The electrodes 111 can be located along the curved section 602 of the lead device 600. In some embodiments, the electrodes 111 can also be located away from the curved section 602 such as distal to the curved section 602. The electrodes 111 can be coupled to a transmission lead 112 extending through the lead device 600.

Material stress can be introduced to the lead device 600 to straighten out the lead device 600 as it is delivered to a target location via a catheter or over a guide wire (not shown). The lead device 600 can be advanced out of the catheter once the catheter is at the target location. The curved section 602 can then deform to its predetermined dimension shape to produce an outward radial force on the cerebral vessel to anchor the lead device 600 in place. For example, once in the target location, the lead device 600 can be advanced from the catheter where it will recoil to its pre-set shape. The material recoil can produce an outward radial force on the cerebral vessel which can anchor the lead device 600 in place. Once the lead device 600 is in place, the one or more electrodes 111 can be activated to stimulate a neural target or record neural signals.

In some embodiments, the lead device 600 can be manufactured such that it can form a “V” shape (for example, in FIG. 6B). By having one or more electrodes 111 positioned at the apex of the “V”, the one or more electrodes 111 can be pushed against the vessel wall. Accordingly, a user can know with more certainty which direction the electrodes 111 will face when implanted. This can allow electrodes 111 to be secured in a specific location and at a specific orientation. In some embodiments, the lead device 600 can be positioned proximal or distal to a target cerebral vessel.

One technical problem faced by the applicant is how to design a lead device 600 that comprises fewer material transition zones and solder or weld joints (all of which increase the manufacturing complexity and introduce unwanted rigid regions that limit the ability of the lead device 600 to navigate tortuous cortical vessels or tight bends). One technical solution discovered and developed by the applicants is to utilize the lead device 600 disclosed herein to carry an electrode array to a target site. The lead device 600 can eliminate the need for material transition zones and solder joints (which improves the manufacturability of the lead device 600) and allow for maximum flexibility and ease of deployment. Furthermore, the lead device 600, when implanted, does not occlude the cerebral vessel lumen and does not adversely affect blood flow within the target vessel.

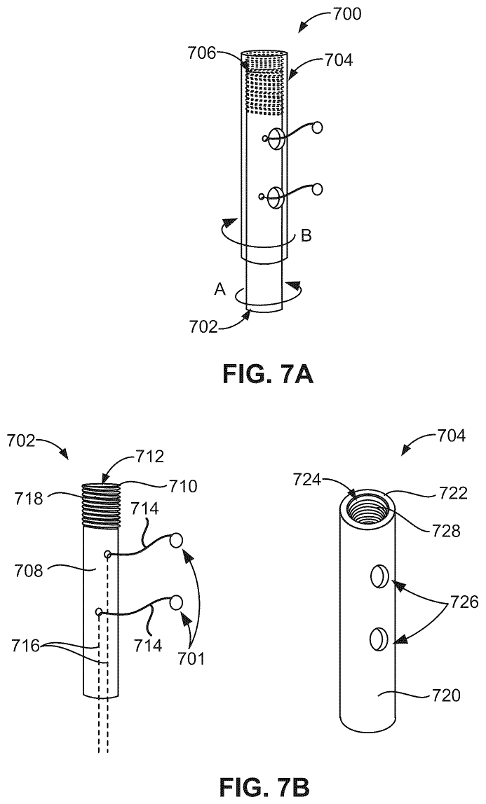

FIG. 7A illustrates a system 700 for implanting a plurality of electrodes 701 within a cerebral vessel (see, e.g., FIG. 25) of a subject. FIG. 7A illustrates the system 700 in an assembled configuration. The system 700 can comprise an inner tubular member 702 and an outer tubular member 704.

FIG. 7B illustrates the inner tubular member 702 and the outer tubular member 702 of the system 700 in a disassembled configuration. The inner tubular member 702 can be configured to detachably couple to the outer tubular member 704 via a threaded connection 706 (see, e.g., FIG. 7A).

The inner tubular member 702 can comprise an inner tubular body 708 having an inner distal end 710 and an inner proximal end (FIG. 7B only shows the portion of the inner tubular member 702 near the inner distal end 710). The inner tubular body 708 can also comprise an inner hollow lumen 712 extending between the inner distal end 710 and the inner proximal end of the inner tubular member 702. The inner tubular member 702 can further comprise the plurality of electrodes 701 extending laterally or substantially orthogonally from the inner tubular body 708 of the inner tubular member 702.

Each of the electrodes 701 can be connected to the tubular body 708 by a conductive wire segment 714 made of a shape memory alloy (e.g., a nickel-titanium wire or Nitinol wire). In other embodiments, the conductive wire segment 714 can be made of another type of conductive material that exhibits shape memory behavior or shape recovery properties. The electrodes 701 can be spaced apart from one another along a length of the inner tubular body 708.

The conductive wire segments 714 can be coupled or connected to conductive wires 716 (shown using broken or phantom lines in FIG. 7B) that extend at least partly through the inner hollow lumen 712. The proximal ends of the conductive wires 716 can be coupled or connected to one or more conductive leads or cables (or serve as parts of conductive leads or cables) for connecting to a telemetry unit or control device.

The inner tubular member 702 can further comprise an exterior threaded pattern 718 (also referred to as a male threaded pattern) defined at the inner distal end 710 of the inner tubular body 708. The exterior threaded pattern 718 can serve as part of the threaded connection 706.

The outer tubular member 704 can be designed or otherwise configured to be compatible with or accommodate the inner tubular member 702. For example, the inner tubular member 702 can fit concentrically within the outer tubular member 704.

The outer tubular member 704 can comprise an outer tubular body 720 having an outer distal end 722 and an outer proximal end (FIG. 7B only shows the portion of the outer tubular member 704 near the outer distal end 722). The outer tubular body 720 can also comprise an outer hollow lumen 724 extending between the outer distal end 722 and the outer proximal end of the outer tubular member 704.

The outer tubular body 720 can comprise a plurality of holes 726 defined along a length of the outer tubular body 720. The holes 726 can be spaced apart from one another. The distance separating the holes 726 can be the same as or approximately equivalent to the distance separating the electrodes 701 of the inner tubular member 702. The holes 726 can allow the electrodes 701 of the inner tubular member 702 to pass through or be exposed. The holes 726 of the outer tubular body 720 can expose the electrodes 701 to an intravascular environment external to the outer tubular member 704.

The outer tubular member 704 can further comprise an interior threaded pattern 728 (also referred to as a female threaded pattern) defined at the outer distal end 722 of the outer tubular body 720. The interior threaded pattern 728 can serve as part of the threaded connection 706 (along with the exterior threaded pattern 718).

FIG. 7A illustrates the system 700 in an assembled configuration with the inner tubular member 702 inserted, at least partly, into the outer tubular member 704 and the exterior threaded pattern 718 of the inner tubular member 702 engaged, at last partly, with the interior threaded pattern 728 of the outer tubular member 704 (e.g., the inner tubular member 702 is partly screwed into the outer tubular member 704). When the system 700 is in the assembled configuration, the electrodes 701 of the system 700 can be fully deployed or fully extended.

Although not shown in FIG. 7A, the system 700 can also have another assembled configuration with the electrodes 701 fully housed, contained, or constricted within the outer hollow lumen 724 of the outer tubular member 704. In this configuration, the inner tubular member 702 is still inserted, at least partly, into the outer tubular member 704 and the exterior threaded pattern 718 of the inner tubular member 702 is still engaged, at last partly, with the interior threaded pattern 728 of the outer tubular member 704. However, in this other assembled configuration, neither the inner tubular member 702 nor the outer tubular member 704 is rotated or twisted to a degree (or to an extent) that allows the electrodes 701 to be deployed out of the holes 726 of the outer tubular member 704. As such, the system 700 in this configuration can be considered an assembled delivery configuration to allow the system 700 to be delivered through tortuous vasculature without the electrodes 701 catching on sensitive vasculature walls during the delivery process. In this configuration, the inner tubular member 702 can translate (rotationally and/or longitudinally) with respect to the outer tubular member 704.

Once the system 700 (in the assembled delivery configuration) is at the implantation site, the inner tubular member 702 can be rotated or twisted to first align the electrodes 701 with the holes 726 and then to push the electrodes 701 through and out of the holes 726 and into the implantation site (e.g., a site within a cerebral vessel). In some embodiments, the electrodes 701 can be pushed or otherwise deployed out of the holes 726 to have the electrodes 701 contact part of a vessel wall (e.g., a cerebral vessel wall).

When the electrodes 701 are aligned with the holes 726, the conductive wire segments 714 can recover part of their preset shape and push or carry the electrodes 701 laterally or orthogonally away from the inner tubular body 708 and out through the holes 726.

In one embodiment, the inner tubular member 702 can be rotated with respect to the outer tubular member 704 in a first rotational direction (shown by rotational arrow A in FIG. 7B) when the system 700 is positioned in a middle of the vessel (i.e., away from the vessel wall). By continuing to rotate the inner tubular member 702, the electrodes 701 can be pushed out further away from the outer tubular member 704 until the electrodes 701 eventually contact the vessel wall.

In another embodiment, the outer tubular member 704 can be rotated with respect to the inner tubular member 702 in a second rotational direction (shown by rotational arrow B in FIG. 7B) opposite the first rotational direction when the system 700 is positioned close to or against a vessel wall. In this embodiment, the electrodes 701 can be exposed or unsheathed by the holes 726 when the electrodes 701 are aligned with the holes 726. Also, in this embodiment, the electrodes 701 can contact the vessel wall but the conductive wire segments 714 are not fully deployed or extended.

The outer tubular body 720 can be sized to fit within a delivery catheter or to navigate through tortuous cerebral vessels without a delivery catheter.

The system 700 can be implanted within a cerebral vessel. The electrodes 701 of the system can be used to record neural activity or stimulate neighboring neural tissue.

In some embodiments, the electrodes 701 can be electrically coupled to a telemetry unit that is implanted within the subject via the conductive wires 716 and/or one or more lead cables. In other embodiments, the electrodes 701 can be electrically coupled to an extracorporeal telemetry unit that is located outside the body of the subject via the conductive wires 716 and/or one or more lead cables.

Although FIGS. 7A and 7B illustrate the system 700 comprising two electrodes 701, it is contemplated by this disclosure and it should be understood by one of ordinary skill in the art that the system 700 can comprise one electrode 701, three electrodes 701, four electrodes 701, five electrodes 701, five electrodes, 701, six electrodes 701, or seven or more electrodes 701.

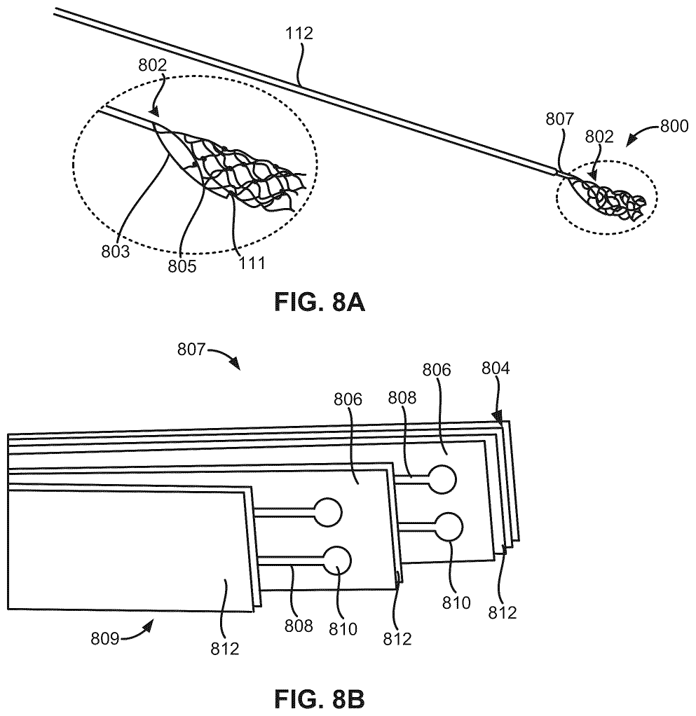

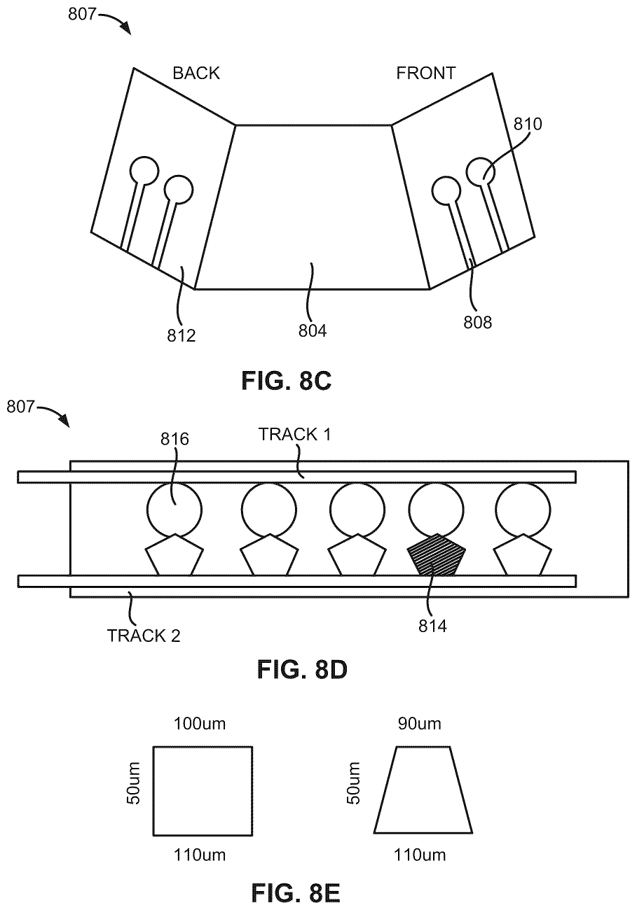

FIG. 8A illustrates one embodiment of a stent 800 configured to be delivered into a cerebral vessel (see, e.g., FIG. 25). The stent 800 can comprise an expandable stent body 802 for stimulating neural targets and recording neural activity within the cerebral vessel. The stent body 802 can be electrically coupled to one or more transmission leads 112.

The stent body 802 can comprise a plurality of struts 803 joined together with a plurality of strut crosslinks 805. The stent body 802 can be in the shape of a tubular lattice or interwoven mesh structure. The stent 800 can comprise a plurality of electrodes 111 coupled to the stent body 802. In some embodiments, the electrodes 111 can be made in part of platinum.

The stent 800 can be implanted within vessels ranging from about 0.5 mm to 5 mm. The stent body 802 can have a diameter between about 0.3 mm to about 1.3 mm, depending on the size of the vessel and the number of electrodes 111 needed to be placed thereon.

The electrodes 111 can comprise a diameter of between about 300 μm and about 750 μm. In some embodiments, the length of the stent 800 can be about 80 mm to about 380 mm.

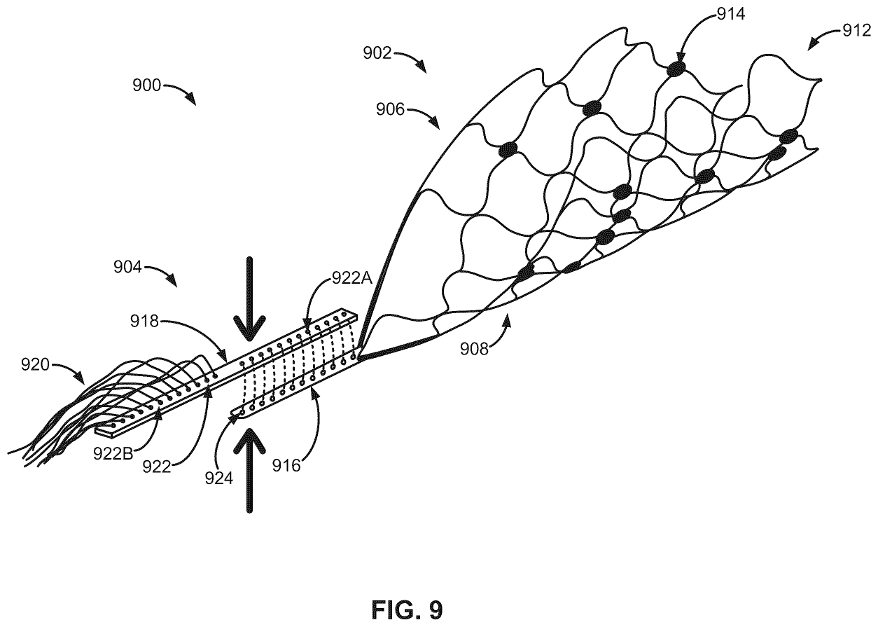

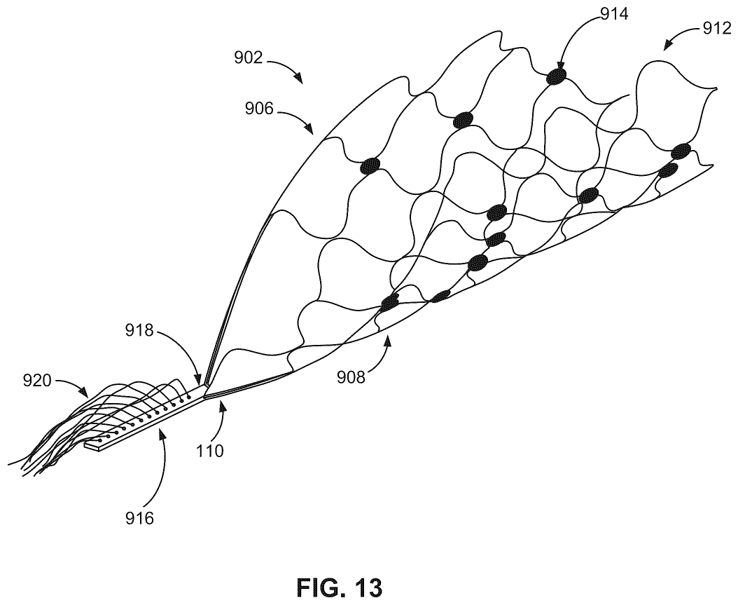

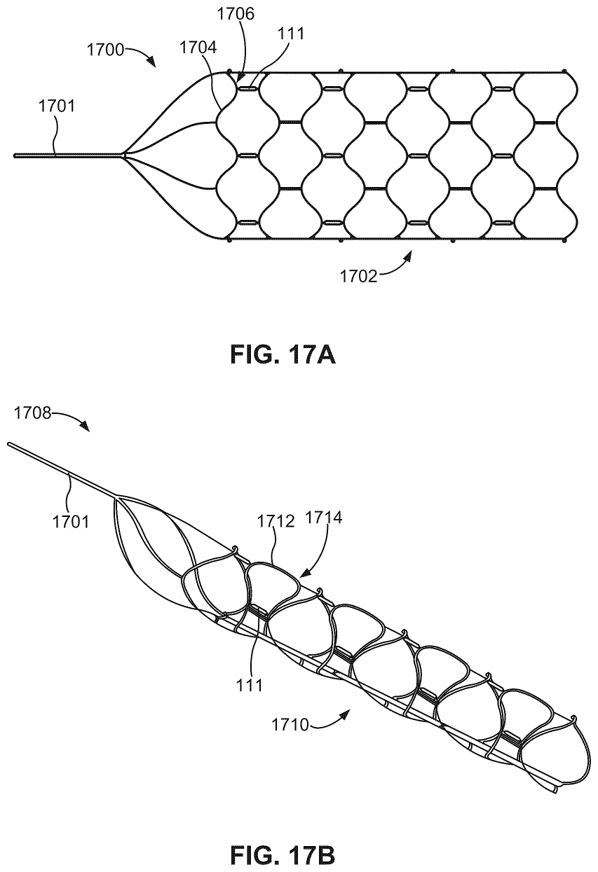

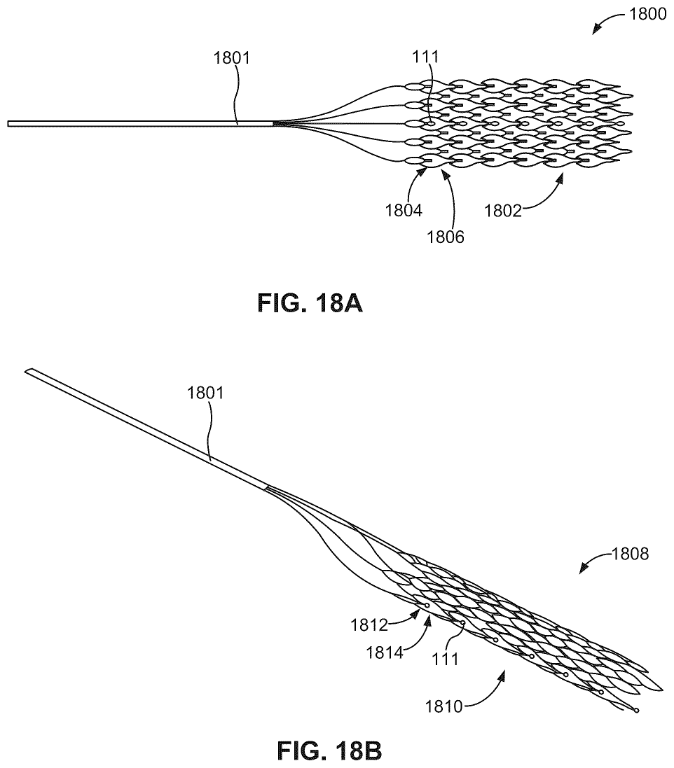

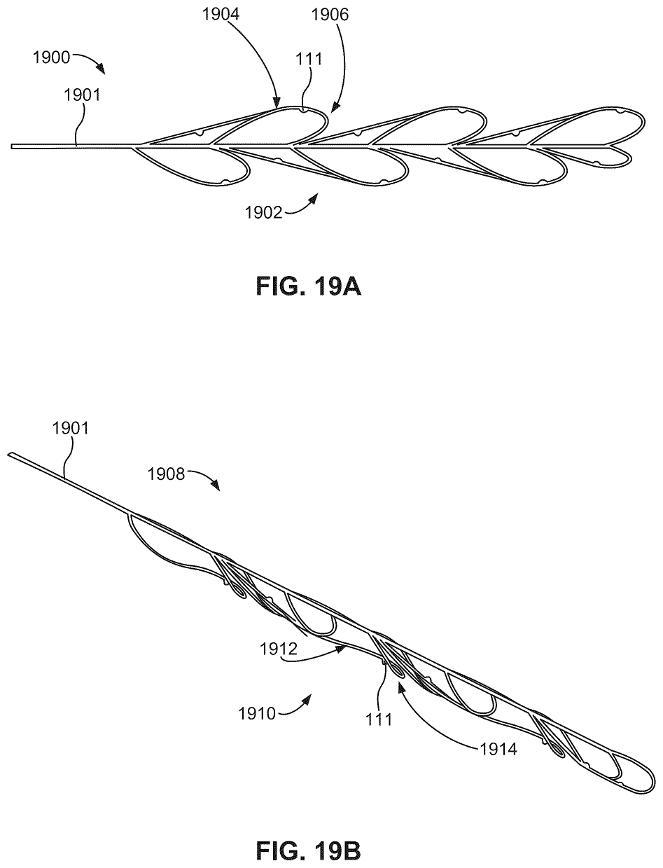

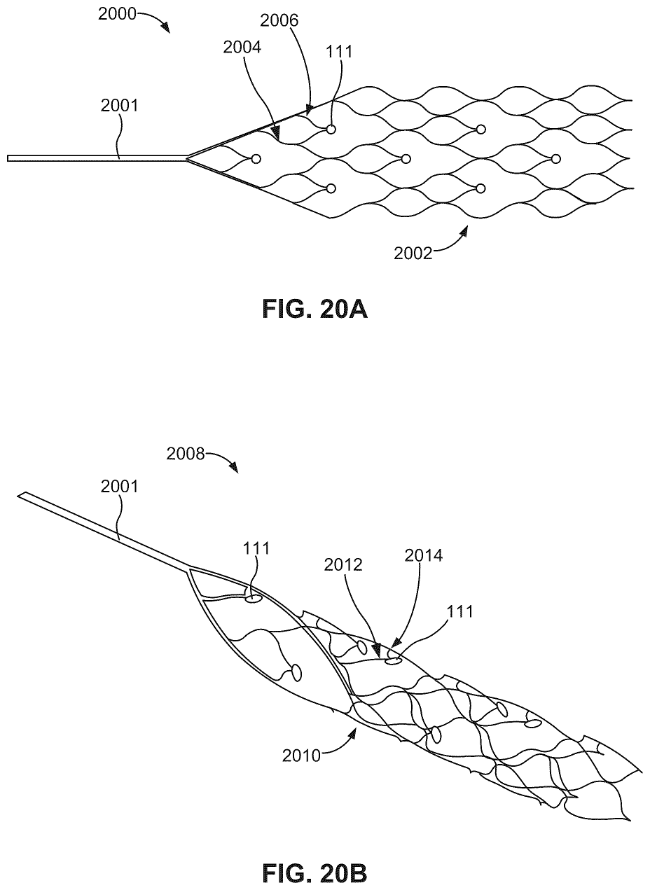

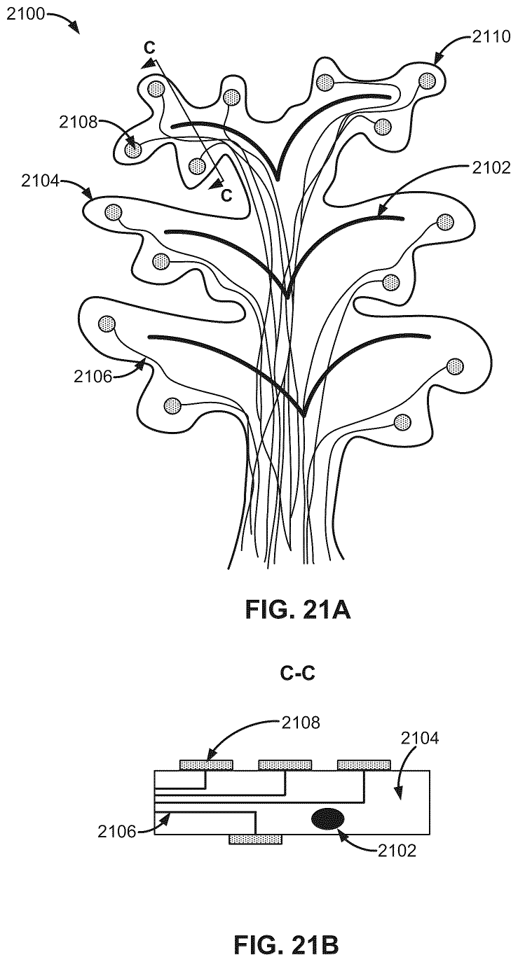

FIG. 8B illustrates various layers 809 of one embodiment of part of a paddle 807 or paddle portion of one of the stents or stent electrode arrays disclosed herein (e.g., stent 800 of FIG. 8A, stent 906 of FIGS. 9 and 13, stent electrode array 1700 of FIG. 17A, stent electrode array 1708 of FIG. 17B, stent electrode array 1800 of FIG. 18A, stent electrode array 1808 of FIG. 18B, stent electrode array 1900 of FIG. 19A, stent electrode array 1908 of FIG. 19B, or stent electrode array 2000 of FIGS. 20A and 20B) comprising stacked conductive tracks 808. The paddle 807 can comprise stacked components such as a flexible base layer 804, one or more insulating layers 806 comprising one or more conductive tracks 808 and conductive pads 810 disposed on the insulating layers 806, and one or more top insulation layers 812.

The base layer 804 can be made of a shape memory alloy. The base layer 804 can have at least one insulating layer 806 disposed on top of the base layer 804. The insulating layer 806 can have one or more conductive tracks 808 and conductive pads 810 disposed on top of the insulating layer 806. Each of the tracks 808 can be electrically coupled to an electrode 111.

The paddle 807 can also have another insulating layer 806 with additional conductive tracks 808 or pads 810 disposed on top of said insulating layer 806. This pattern can be repeated as necessary (i.e., a first insulation layer, a second insulation layer, and a third insulation layer with first and second conductive tracks therebetween). The paddle 807 can be connected to certain conductive wires or wire bundles disclosed herein.

The stacked arrangement of the paddle 807 can allow the stents or stent electrode arrays to have more electrodes 111. Also, the stacked arrangement of the paddle 807 can allow the paddle to be easily inserted into a microcatheter having a catheter diameter of about 500 μm.

The paddle 807 can connect a stent body (e.g., stent body 802) to a transmission lead 112. The transmission lead 112 can be electrically coupled or otherwise connected to a telemetry unit implanted within a subject (not shown).

In some embodiments, the stent 800 can comprise a plurality of electrode mounting sites scattered along the stent body 802. Similar to the paddle 807, at least part of the stent 800 can also be made of the base layer 804, a first insulating layer (e.g., a first instance of the insulating layer 806) stacked on top of the base layer 804, a first conductive track (e.g., a first instance of the conductive track 808) disposed on the first insulating layer, a second insulating layer (a second instance of the insulating layer 806) stacked on top of the first conductive track and the first insulating layer, a second conductive track (e.g., a second instance of the conductive track 808) disposed on the second insulating layer, and a third insulating layer (a third instance of the insulating layer 806) stacked on top of the second conductive track and the second insulating layer.

The base layer 804 can be made in part of a nickel-titanium alloy. The base layer 804 can have a thickness of between about 40 μm to about 60 μm.

At least one of the first insulating layer, the second insulating layer, and the third insulating layer can have a thickness of between about 200 nm and about 400 nm.

At least one of the first conductive track and the second conductive track can be made in part of platinum. At least one of the first conductive track and the second conductive track can have a thickness of between about 200 nm and about 400 nm.

At least one of the first conductive track and the second conductive track can have a width of at least about 30 μm. At least one of the first conductive track and the second conductive track can be separated from an edge of the strut by at least 27 μm.

At least one of the first conductive track and the second conductive track can have a track cross-section. The track cross-section can be substantially shaped as a trapezoid.

In some embodiments, the stacked components can comprise a 50 μm Nitinol base layer, one or more 300 nm Yttrium stabilized zirconia (YSZ) insulation layers, and one or more 300 nm pure platinum tracks.

FIG. 8C illustrates a variation of part of a paddle 807 comprising stacked tracks 808 to enable connection on both sides of a base layer 804. The base layer 804 or scaffold can therefore comprise tracks layered on both a front and a back of the paddle 807 accordingly.

In some embodiments, connections can be made to both a top and a bottom of the paddle 807. Connections to two sides can reduce the width of the paddle 807 and/or increase the electrode count.

FIG. 8D illustrates a variation of part of a paddle 807 comprising two tracks connected to a plurality of electrodes. The paddle 807 can comprise a plurality of paddle connections. One or more on-electrode elements 814 and one or more off-electrode elements 816 can determine which of the electrodes is to be on and which are to remain off for sending a stimulation current or receiving neural activity.

FIG. 8E illustrates various cross-sectional areas of tracks or layers of part of a strut of a stent (e.g., stent 800 of FIG. 8A, stent 906 of FIGS. 9 and 13, stent electrode array 1700 of FIG. 17A, stent electrode array 1708 of FIG. 17B, stent electrode array 1800 of FIG. 18A, stent electrode array 1808 of FIG. 18B, stent electrode array 1900 of FIG. 19A, stent electrode array 1908 of FIG. 19B, or stent electrode array 2000 of FIGS. 20A and 20B). In one embodiment, the strut can have a rectangular cross-section with a width of about 110 μm and a height of about 50 μm.

In other embodiments, the strut can have a trapezoidal cross-section with a base width of about 110 μm, a top width of about 90 μm, and a height of about 50 μm.

FIG. 9 illustrates a variation of an implantable electrode array 900 for recording neural activity and/or stimulating neural tissue within a brain, a subarachnoid space, or a cranial extravascular location of a subject.

In some embodiments, the implantable carrier 902 can be an implantable endovascular carrier. The implantable electrode array 900 can be designed for delivery into small cerebral vessels. The implantable electrode array 900 can also be implanted in other bodily vessels of the subject.

The implantable electrode array 900 can comprise an implantable carrier 902 and an electrical interface 904. As shown in FIG. 9, the implantable carrier 902 can be implemented as an implantable stent 906 or scaffold comprising a carrier body (e.g., a stent body 908) comprising a carrier proximal end (e.g., a stent proximal end 910) and a carrier distal end (e.g., a stent distal end 912). The implantable carrier 902 (e.g., the implantable stent 906) can also comprise a plurality of electrodes 914 coupled to various positions along the carrier body (e.g., the stent body 908).

The implantable carrier 902 (e.g., the stent 906) can also comprise a paddle 916 extending from the carrier proximal end (e.g., the stent proximal end 910) of the carrier body (e.g., the stent body 908).

In some embodiments, the stent 906 can comprise a plurality of struts coupled together with a plurality of crosslinks. In certain embodiments, the stent body 908 can be in the shape of a tubular lattice or interwoven mesh structure. The stent body 908 can be expandable or self-expandable. When the stent body 908 is in an expanded configuration or form, the plurality of electrodes 914 can be pressed against or otherwise contact a cerebral vessel wall in order to record neural activity or stimulate certain neural targets. The stent body 908 can also be collapsible.

The electrical interface 904 of the implantable electrode array 900 can be a flexible printed circuit 918. The flexible printed circuit 918 can be coupled to the paddle 916 of the stent 906.

Also, as shown in FIG. 9, the electrical interface 904 can be implemented as an elongated flexible printed circuit strip. For example, the electrical interface 904 can be implemented as a substantially rectangular elongated flexible printed circuit strip.

In other embodiments, the electrical interface 904 can be implemented as a substantially elongated ovoid or triangular flexible printed circuit. The electrical interface 904 can take on other shapes as long as the shape complements or matches the shape of the paddle 916 of the implantable carrier 902.

FIG. 9 also illustrates that the electrical interface 904 can comprise one or more conductive wires 920 coupled to the flexible printed circuit 918.

The flexible printed circuit 918 serving as the electrical interface 904 can also comprise a plurality of through-holes 922 including a first set of through-holes 922A and a second set of through-holes 922B. The first set of through-holes 922A can be positioned distal to the second set of through-holes 922B along the flexible printed circuit 918. For example, the first set of through-holes 922A can be positioned closer to the implantable carrier 902 than the second set of through-holes 922B.

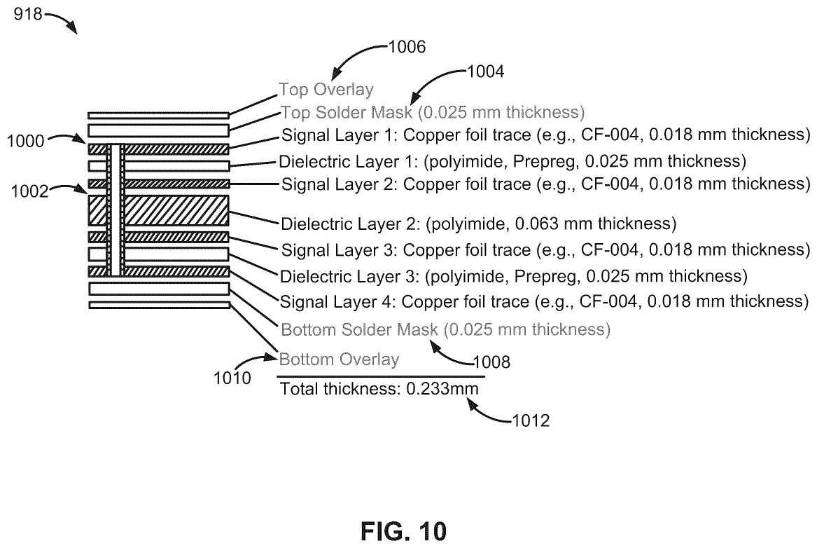

FIG. 10 is a side cross-sectional view of part of the flexible printed circuit 918. As previously discussed, the electrical interface 904 of the implantable electrode array 900 can comprise the flexible printed circuit 918. As shown in FIG. 10, the flexible printed circuit 918 can comprise a plurality of layers stacked on top of one another.

The flexible printed circuit 918 can comprise a plurality of signal layers 1000 comprising conductive traces (e.g., copper foil traces). In some embodiments, the flexible printed circuit 918 can comprise two signal layers 1000, three signal layers 1000, or four or more signal layers 1000.

The conductive traces can be made of gold, platinum, or another conductive material. The signal layers 1000 of the flexible printed circuit 918 can be separated by one or more dielectric layers 1002.

Each of the signal layers 1000 can have a signal layer thickness. In some embodiments, the signal layer thickness can be between about 0.010 mm and 0.020 mm (about 0.018 mm).

In some embodiments, the dielectric layers 1002 can be made in part of polyimide. In certain embodiments, at least one of the dielectric layers 1002 can be a polyimide-based prepreg layer. In other embodiments, at least one of the dielectric layers 1002 can be a non-prepreg polyimide layer.

The dielectric layers 1002 can each have a dielectric layer thickness. In some embodiments, the dielectric layer thickness can be between about 0.020 mm and 0.070 mm. As a more specific example, the dielectric layer thickness can be between about 0.025 mm and 0.063 mm. As a more specific example, a non-prepreg polyimide layer can be thicker than two thinner polyimide-based prepreg sandwiching the thicker non-prepreg polyimide layer.

FIG. 10 also illustrates that the signal layers 1000 can comprise a top-most signal layer (e.g., signal layer 1) and a bottom-most signal layer (e.g., signal layer 4). The top-most signal layer can be covered by a top solder mask layer 1004 and a top overlay 1006.

The bottom-most signal layer can be covered by a bottom solder mask layer 1008 and a bottom overlay 1010.

The flexible printed circuit 918 can have a total thickness 1012 of between 0.20 mm and 0.25 mm. The total thickness 1012 can be a thickness of all of the layers combined. For example, the flexible printed circuit 918 can have a total thickness 1012 of about 0.233 mm. The flexible printed circuit 918 can have a total thickness 1012 of less than about 0.250 mm.

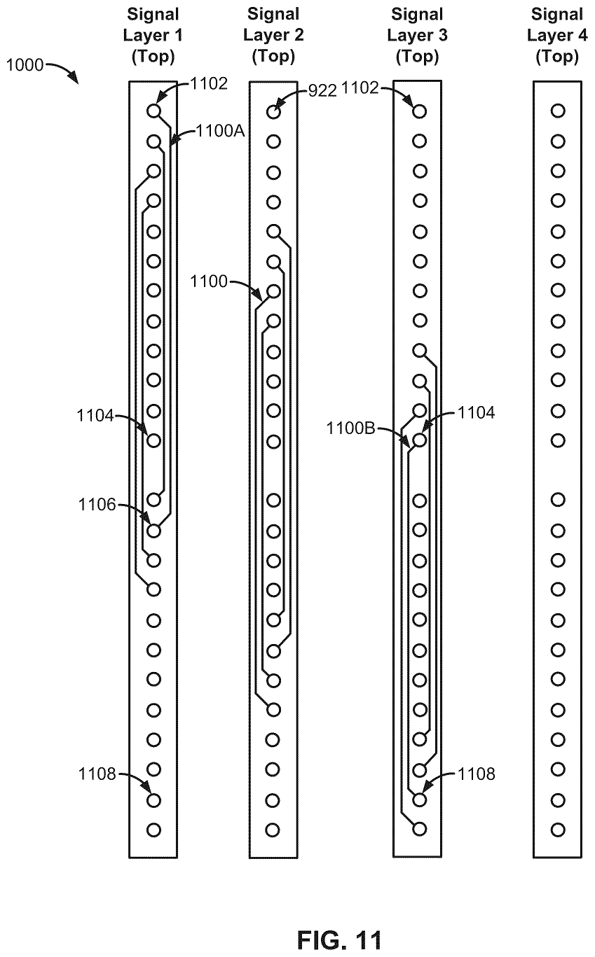

FIG. 11 illustrates a top view of a plurality of signal layers 1000 of one embodiment of the flexible printed circuit 918 (see, e.g., FIGS. 9 and 10). As shown in FIG. 11, at least one of the through-holes of the first set of through-holes 922A can be electrically coupled to at least one of the through-holes of the second set of through-holes 922B via a conductive trace 1100. The conductive trace 1100 can be part of one of the signal layers 1000.

In some embodiments, each of the through-holes of the first set of through-holes 922A can be electrically coupled to one of the through-holes of the second set of through-holes 922B via a conductive trace 1100.

In some embodiments (for example, in the embodiment shown in FIG. 11), each signal layer 1000 of the flexible printed circuit 918 can comprise conductive traces 1100 that electrically couple a subset or some (but not all) of the through-holes of the first set of through-holes 922A with a subset or some (but not all) of the through-holes of the second set of through-holes 922B.

For example, as shown in FIG. 11, the first set of through-holes 922A can comprise a first distal-most through-hole 1102 and a first proximal-most through-hole 1104. Moreover, the second set of through-holes 922B can comprise through-hole 1106 and through-hole 1108. The first distal-most through-hole 1102 can be electrically coupled to the through-hole 1106 via a conductive trace (for example, conductive trace 1100A) of a signal layer 1000 (for example, signal layer 1). Also, the first proximal-most through-hole 1104 can be electrically coupled to the through-hole 1108 via a separate conductive trace (for example, conductive trace 1100B) of another signal layer 1000 (for example, signal layer 3).

As previously discussed, the signal layers 1000 can be stacked on top of one another and be separated by dielectric layers. Although FIG. 11 illustrates four signal layers and twenty-four through-holes 922, it is contemplated by this disclosure that the number of signal layers can be between two and four or greater than four and the number of through-holes 922 can be less than twenty-four or greater than twenty-four.

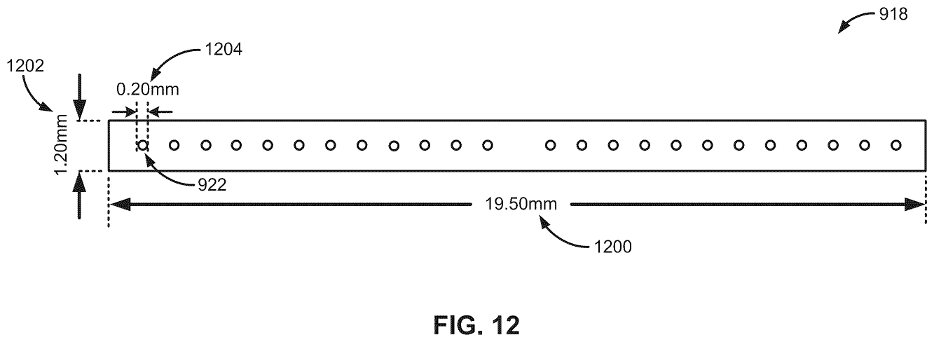

FIG. 12 illustrates that the flexible printed circuit 918 can be implemented as a substantially elongated rectangular flexible printed circuit strip. In these embodiments, the flexible printed circuit strip can have a strip length 1200 and a strip width 1202.

In some embodiments, the strip length 1200 can be between about 15.0 mm to about 20.0 mm (e.g., about 19.50 mm).

In some embodiments, the strip width 1202 can be between about 1.0 mm and 2.0 mm (e.g., about 1.20 mm).

FIG. 12 also illustrates that each of the through-holes 922 can have a hole diameter 1204. In some embodiments, the hole diameter 1204 can be between 0.10 mm and 0.30 mm (e.g., about 0.20 mm).

Referring back to FIG. 9, the conductive wires 920 can be coupled to the signal layers 1000 via the second set of through-holes 922B. This can provide a structural benefit as bonding the conductive wires 920 directly to a surface of a signal layer means that the bond strength depends wholly on the bonding method in the presence of a shear force. However, if the conductive wires 920 are passed through one or more layers before being coupled to the signal layers 1000, some of that shear force can be transmitted to a cross-section of the wire as the wire bends through the one or more layers.

In some embodiments, at least one conductive wire 920 can be coupled to one of the signal layers 1000 via one of the second set of through-holes 922B.

Each of the conductive wires 920 can comprise a wire distal end and a wire proximal end. The wire distal end can be coupled to the flexible printed circuit 918. The wire proximal end can be connected to a lead cable or directly to a telemetry unit.

In some embodiments, the lead cable can be coupled to the telemetry unit used to obtain or record signals from the plurality of electrodes 914. In certain embodiments, the telemetry unit can also be used to generate pulses for stimulating neural tissue via the electrodes 914.

As shown in FIG. 9, the paddle 916 can comprise a conductive epoxy 924 disposed on one side (e.g., a top side) of the paddle 916. The first set of through-holes 922A can be aligned with the conductive epoxy 924 when the flexible printed circuit 918 is coupled to the paddle 916. In some embodiments, the conductive epoxy 924 can be a conductive silver epoxy.

In certain embodiments, each of the electrodes 914 on the stent body 908 can be electrically coupled to one of the first set of through-holes 922A via the conductive epoxy 924 and conductive traces extending through the stent body 908.

The conductive epoxy 924 can act as an adhesive. The conductive epoxy 924 can fill the first set of through-holes 922A to create a conductive connection.

For example, each drop of the conductive epoxy 924 (e.g., silver epoxy) can be positioned on a point of contact with an electrical trace from the stent body 908.

In some embodiments, the paddle 916 can be coupled to the flexible printed circuit 918 via an adhesive. In certain embodiments, the adhesive can be liquid epoxy resin. For example, the liquid epoxy resin can comprise bisphenol A diglycidyl ether. As a more specific example, the adhesive can be a biocompatible EPO-TEK™ epoxy (e.g., EPO-TEK™ MED-301).

In some embodiments, the thickness of the paddle 916 can be between about 0.025 mm and about 0.30 mm (e.g., between about 0.05 mm and about 0.10 mm). The paddle 916 can have a paddle length. In some embodiments, the paddle length can be between about 8.0 mm and 10.0 mm.

For example, the paddle 916 can be made of a stack of materials including a Nitinol base layer, a first yttria-stabilized zirconia (YSZ) insulation layer above the Nitinol base layer, a layer of platinum traces above the first YSZ insulation layer, and a second YSZ insulation layer disposed above the platinum trace layer.

In some embodiments, the paddle 916 of FIG. 9 can be constructed in the same manner as paddle 807 of FIGS. 8A and 8B.

One technical problem faced by the applicant is how to attach a delicate and thin implantable electrode array to rigid cables or wires. This abrupt transition at the interface between the fragile and thin implantable electrode array can be subject to increased mechanical stress and result in connection failure. This can also lead to low yield when manufacturing the implantable electrode array. One technical solution discovered and developed by the applicant is the electrical interface disclosed herein made in part of a flexible printed circuit. The electrical interface can be coupled to a part of the delicate and thin implantable electrode array (for example, a paddle of the implantable electrode array) via an adhesive. The additional flexibility afforded by the flexible printed circuit of the electrical interface can allow the flexible printed circuit to more easily adhere to the delicate and thin implantable electrode array. The flexible printed circuit can comprise a first set of through-holes and a second set of through-holes positioned proximal to the first set of through-holes. The flexible printed circuit can also comprise a plurality of conductive signal layers stacked on top of one another and separated by dielectric layers. The first set of through-holes can be electrically coupled to the second set of through-holes via conductive traces of the conductive signal layers. A conductive epoxy can also be used to connect the first set of through-holes with conductive contacts arranged along part of the implantable electrode array (for example, the paddle of the electrode array). One or more stiffer conductive wires or cables can be coupled to the flexible printed circuit via the second set of through-holes using more traditional attachment techniques with a reduced risk of failure.

FIG. 13 illustrates that the conductive wires 920 can be directly coupled to the flexible printed circuit 918 coupled to the paddle 916 of an implantable electrode array. As previously discussed, the flexible printed circuit 918 can comprise a plurality of signal layers stacked on top of one another (see, e.g., FIG. 10).

For example, the conductive wires 920 can be coupled to a signal layer (see, e.g., FIG. 10) of the flexible printed circuit 918 via a set of through-holes.

In other embodiments, the conductive wires 920 can be directly coupled to the paddle 916 of the implantable carrier 902 (e.g., stent 906).

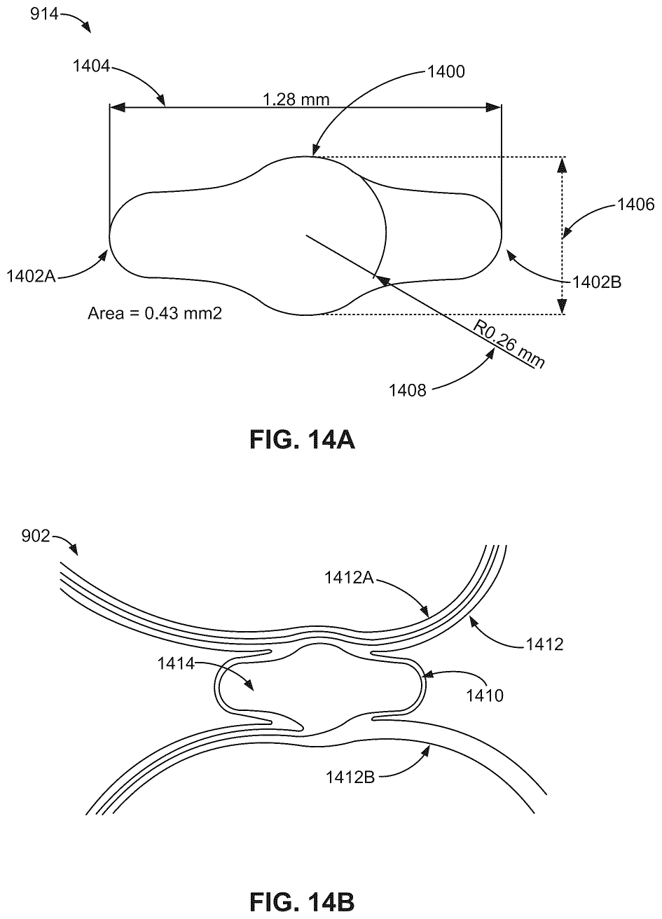

FIG. 14A illustrates one embodiment of an electrode 914 of the implantable electrode array 900. As shown in FIG. 14A, the electrode 914 can have a flattened ovoid shape with an enlarged circular midsection 1400. The electrode 914 can also be considered to have a dual pyriform (or pear) shape where the two widened bases of the pyriform shapes overlap to form the enlarged circular midsection 1400 of the dual pyriform shape.

The electrode 914 can have a first longitudinal end 1402A and a second longitudinal end 1402B. Both the first longitudinal end 1402A and the second longitudinal end 1402B can be rounded or curved. The electrode 914 can also have an electrode length 1404 as measured from the first longitudinal end 1402A to the second longitudinal end 1402B.

As shown in FIG. 14A, the rounded or curved sides of the enlarged circular midsection 1400 of the electrode 914 can define the lateral-most sides of the electrode 914. The electrode 914 can also have an electrode width 1406 as measured from one lateral-most end of the enlarged circular midsection 1400 of the electrode 914 to the opposite (diametrically-opposite) end of the enlarged circular midsection 1400.

In some embodiments, the electrode length 1404 can be greater than the electrode width 1406. For example, the electrode length 1404 can be between about 1.00 mm and about 2.00 mm. As a more specific example, the electrode length 1404 can be about 1.28 mm.

In some embodiments, the electrode width 1406 can be between about 0.40 mm and about 0.60 mm. As a more specific example, the electrode length 1404 can be about 0.52 mm.

The enlarged circular midsection 1400 of the electrode 914 can also have a midsection radius 1408. In some embodiments, the midsection radius 1408 can be between about 0.20 mm and about 0.40 mm. As a more specific example, the midsection radius 1408 can be about 0.26 mm.

In some embodiments, the electrode 914 can be made of a noble metal or an alloy or composite thereof. For example, the electrode 914 can be made of platinum.

In other embodiments, the electrode 914 can be made of platinum black, gold, iridium, palladium, rhodium, a gold-palladium alloy, or a platinum-iridium alloy.

In some embodiments, the implantable electrode array 900 can comprise between seven and twelve electrodes 914. For example, the implantable electrode array 900 can comprise about eleven electrodes 914.

FIG. 14B illustrates that the electrode 914 shown in FIG. 14A can be placed or positioned within an electrode window 1410 arranged between adjacent struts 1412 of the implantable carrier 902.

The implantable carrier 902 can be any of the carriers 902 shown in FIGS. 9 and 13.

In other embodiments, the electrode 914 shown in FIG. 14A can also replace any of the electrodes 2108 shown in FIG. 21A below.

As depicted in FIG. 14B, the electrode window 1410 can be positioned between a first strut 1412A and a second strut 1412B. In certain embodiments, the electrode window 1410 can be positioned or defined along a cross-link connecting neighboring or adjacent struts 1412.

In some embodiments, one or more insulating layers can be used to define at least part of the electrode window 1410. In these and other embodiments, the insulating layers can secure the electrode 914 to the implantable carrier 902.

As shown in FIG. 14B, the electrode window 1410 can be shaped to accommodate the electrode 914. For example, the electrode window 1410 can be shaped as an oval having an enlarged circular midsection. The electrode window 1410 can also be considered to have a dual pyriform (or pear) shape where the two widened bases of the pyriform shapes overlap to form the enlarged circular midsection of the dual pyriform shape.

In some embodiments, the struts 1412 can be made of a shape memory alloy such as a nickel-titanium alloy (e.g., Nitinol). In other embodiments, the struts 1412 can be made of stainless steel, platinum, gold, or alloys or composites thereof.

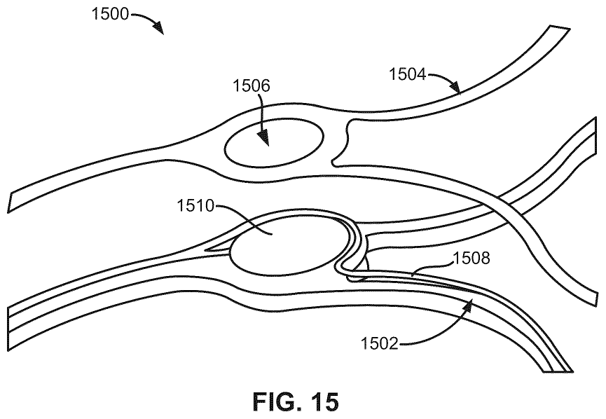

FIG. 15 illustrates a close-up view of an electrode mounting site 1500 of one of the stents or stent electrode arrays disclosed herein (e.g., stent 800 of FIG. 8A, stent 906 of FIGS. 9 and 13, stent electrode array 1700 of FIG. 17A, stent electrode array 1708 of FIG. 17B, stent electrode array 1800 of FIG. 18A, stent electrode array 1808 of FIG. 18B, stent electrode array 1900 of FIG. 19A, stent electrode array 1908 of FIG. 19B, or stent electrode array 2000 of FIGS. 20A and 20B).

The electrode mounting site 1500 can comprise a plurality of struts 1502 covered by an insulation layer 1504. An electrode window 1506 can be formed along the insulation layer 1504. One or more conductive traces 1508 (or conductive tracks) can be disposed on the struts 1502. The conductive traces 1508 can be covered by the insulation layer 1504. The electrode window 1506 can expose an electrode 1510 (e.g., a platinum electrode). Although not shown in FIG. 15, another conductive trace 1508 can be disposed on the insulation layer 1504 with this conductive trace 1508 covered by another insulation layer 1504. Additional conductive trace(s) 1508 and insulation layers 1504 can be further disposed on the top-most insulation layer 1504.

The struts 1502 can be made in part of Nitinol (e.g., Nitinol wire), stainless steel, gold, platinum, nickel, titanium, tungsten, aluminum, nickel-chromium alloy, gold-palladium-rhodium alloy, chromium-nickel-molybdenum alloy, iridium, rhodium, or a combination thereof. The struts 1502 of can also be made in part of a shape memory polymer.

One technical problem faced by the applicant is how to increase the number of electrodes 1510 without decreasing the width of the conductive traces 1508 since each electrode 1510 requires an individual conductive trace 1508 laid next to it. Decreasing the width of a conductive trace 1508 may result in an increase in the resistance of the conductive trace 150 (thereby increasing noise interference). One technical solution discovered and developed by the applicant is to design a stent or stent electrode array with a plurality of multi-layered electrode mounting sites 1500 that can allow the stent or stent electrode array to carry more electrodes.

A minimum trace width can be 30 μm with a 10 μm separation between the traces 1508. The distance between the traces 1508 can be 27 μm.

FIG. 16A illustrates one embodiment of an unraveled electrode array 1600. The unraveled electrode array 1600 can comprise a plurality of wires 1602 connected at the proximal ends of such wires 1602 to a transmission lead 1608.

FIG. 16B illustrates a close-up view of part of the unraveled electrode array 1600. In some embodiments, the wires 1602 can each comprise a distal wire segment 1604 and a distal wire end 1606 serving as a distal terminus of the wire 1602. The distal wire segment 1604 of each of the wires 1602 can protrude out of a peripheral or central lumen beyond a distal end of a tubular body 1610 of the unraveled electrode array 1600.

At least one electrode 111 can be coupled to the distal wire end 1606 of each of the wires 1602. The distal wire segments 1604 can be unraveled, unbraided, or unwound such that the unraveled, unbraided, or unwound distal wire segments 1604 appear as elongated fingers.

The plurality of wires 1602 can comprise a first wire comprising a first distal wire segment 1604A having a first distal wire segment length. The plurality of wires can also comprise a second wire comprising a second distal wire segment 1604B having a second distal wire segment length. The first distal wire segment length can be greater than the second distal wire segment length.

Each of the wires 1602 can comprise an inner conductive wire surrounded by an insulating coating or layer. The electrode 111 can be electrically coupled to the inner conductive wire at the distal wire end 1606.

In some embodiments, each of the distal wire segments 1604 can be coupled to its own electrode 111 to reduce any instances of electrode cross-talk and electrode shunting since the electrodes 111 are spaced apart from one another.

In alternative embodiments, each of the distal wire segments 1604 can have more than one electrode 111 coupled to the distal wire end 1606 of the distal wire segment 1604.

In some embodiments, the distal wire segments 1604 can have varying thicknesses. This can allow for different forces to be applied to vessel walls at varying radial forces for purposes of better wall apposition and anchoring. In some embodiments, the distal wire segments 1604 can have varying lengths and, as such, can advance into multiple vessels simultaneously, including small vessels and branching vessels that are opposite to each other with respect to the superior sagittal sinus (e.g., at least distal wire segment 1604 can extend into the left hemisphere of the brain and at least one other distal wire segment 1604 can extend into the right hemisphere of the brain).

Another advantage of the unraveled electrode array 1600 is that the unraveled wires 1602 can prevent the electrodes 111 from clumping together when the unraveled electrode array 1600 navigates through small tortuous vessels.

FIG. 17A illustrates one embodiment of a stent electrode array 1700 comprising a substantially tubular stent body 1702 comprising struts 1704 forming a plurality of undulating rings 1706 and electrodes 111 serving as connectors connecting the undulating rings 1706.

In some embodiments, the tubular stent body 1702 can be made in part of a shape memory material such as Nitinol. A proximal end of the tubular stent body 1702 can be coupled to a paddle 1701. The paddle 1701 can be constructed similar to paddle 807 (see, e.g., FIG. 8A) or paddle 916 (see, e.g., FIG. 9) and operate in a similar manner.

FIG. 17B illustrates another embodiment of a stent electrode array 1708 comprising a substantially tubular stent body 1710 comprising struts 1712 forming a plurality of deformed hoops 1714 and electrodes 111 serving as connectors connecting part of the deformed hoops 1714.

The geometry/design of the stent electrode array 1700 and the stent electrode array 1708 can allow for electrodes 111 with larger electrode surface areas. For example, electrodes 111 can be made longer (e.g., about 1 mm to about 3 mm long), which can allow these electrodes 111 to contact more neural tissue. In addition, the symmetry of this design can also improve fatigue resistance and deliverability.

FIG. 18A illustrates another embodiment of a stent electrode array 1800 comprising a substantially tubular stent body 1802 comprising struts 1804 forming a plurality of piriform structures 1806 and electrodes 111 serving as ends or tips of the piriform structures 1806.

As shown in FIG. 18A, the electrodes 111 can be aligned in a linear fashion or aligned longitudinally (e.g., in a row) with respect to the tubular stent body 1802 coupled to the stent electrode array 1800.

In some embodiments, the tubular stent body 1802 can be made in part of a shape memory material such as Nitinol. A proximal end of the tubular stent body 1802 can be coupled to a paddle 1801. The paddle 1801 can be constructed similar to paddle 807 (see, e.g., FIG. 8A) or paddle 916 (see, e.g., FIG. 9) and operate in a similar manner.

FIG. 18B illustrates another embodiment of a stent electrode array 1808 comprising a substantially tubular stent body 1810 comprising struts 1812 forming a plurality of pointed oval structures 1814 and electrodes 111 serving as ends or tips of the pointed oval structures 1814.

The stent body 1810 can comprise electrodes 111 aligned in a linear fashion or aligned longitudinally (e.g., in a row) with respect to the tubular stent body 1702 coupled to the stent electrode array 1808.

In some embodiments, the tubular stent body 1810 can be made in part of a shape memory material such as Nitinol. A proximal end of the tubular stent body 1810 can be coupled to a paddle 1801. The paddle 1801 can be constructed similar to paddle 807 (see, e.g., FIG. 8A) or paddle 916 (see, e.g., FIG. 9) and operate in a similar manner.

The geometry/design of the stent electrode array 1800 and the stent electrode array 1808 can allow for the electrodes 111 to be positioned on one side of the stent body (e.g., the stent body 1802 or the stent body 1810). This can allow the opposite side of the stent body (e.g., the stent body 1802 or the stent body 1810) to be made of more stent body material (e.g., shape memory material such as Nitinol). This can provide for greater radiopacity of the opposite side of the stent body and knowledge regarding which orientation the electrodes 111 are facing. This can allow a physician or other medical professional to implant the stent electrode array (either the stent electrode array 1800 or the stent electrode array 1808) in an orientation that allows the electrodes 111 to face a neural stimulation target or face a neural recording site.

FIG. 19A illustrates another embodiment of a stent electrode array 1900 comprising a stent body 1902 comprising struts 1904 forming alternating leaf-like wire structures 1906 and electrodes 111 coupled to the leaf-like wire structures 1906.

In some embodiments, the stent body 1902 can be made in part of a shape memory material such as Nitinol. A proximal end of the stent body 1902 can be coupled to a paddle 1901. The paddle 1901 can be constructed similar to paddle 807 (see, e.g., FIG. 8A) or paddle 916 (scc, e.g., FIG. 9) and operate in a similar manner.

FIG. 19B illustrates another embodiment of a stent electrode array 1908 comprising a stent body 1910 comprising struts 1912 forming collapsed hoops 1914 and electrodes 111 coupled to the collapsed hoops 1914. The electrodes 111 can be pushed against a vessel wall via the hoops 1914 when expanded. The individual hoops 1914 can enable the stent electrode array 1900 to be placed in vessels of varying and irregular internal diameter.

In some embodiments, the stent body 1910 can be made in part of a shape memory material such as Nitinol. A proximal end of the stent body 1910 can be coupled to a paddle 1901. The paddle 1901 can be constructed similar to paddle 807 (see, e.g., FIG. 8A) or paddle 916 (see, e.g., FIG. 9) and operate in a similar manner.

The stent electrode array 1900 and the stent electrode array 1908 can be made of less material, increasing the safety of the stent electrode arrays as a result of the reduced implanted metal. The stent electrode array 1900 and the stent electrode array 1908 can also be pushed forward without buckling on itself due to the stent body extending nearly to the end of the entire stent electrode array.

The stent bodies disclosed herein can be laser cut or woven in a manner such that there is additional material or markers where the electrodes 111 can be placed to assist with the attachment of the electrodes 111.

Alternatively, the stent bodies disclosed herein can be manufactured using thin-film technology. For example, material (e.g., shape memory alloys such as Nitinol, conductive polymers, other non-shape memory alloys, or biocompatible metals such as platinum, iridium, stainless steel, gold, or a combination thereof) can be deposited in specific locations to grow or build the stent structures disclosed herein layer by layer.

The stent bodies can also be fabricated such that the stent structure comprises an integrated conductive layer that extends through a portion of the stent and where the electrodes 111 are coupled to an exposed portion of the integrated conductive layer.

FIG. 20A illustrates another embodiment of a stent electrode array 2000 comprising a substantially tubular stent body 2002 comprising struts 2004 forming a plurality of limoniform structures 2006 and electrodes 111 serving as ends of the limoniform structures 2006. The stent electrode array 2000 design can allow electrodes 111 to be less impacted by changes and irregularities in the vessel diameter as the electrodes 111 can protrude further out than a typical stent shape.

In some embodiments, the stent body 2002 can be made in part of a shape memory material such as Nitinol. A proximal end of the stent body 2002 can be coupled to a paddle 2001. The paddle 2001 can be constructed similar to paddle 807 (see, e.g., FIG. 8A) or paddle 916 (see, e.g., FIG. 9) and operate in a similar manner.

FIG. 20B illustrates an embodiment of a stent electrode array 2008 comprising a substantially tubular stent body 2010 comprising struts 2012 forming a plurality of deformed or collapsed limoniform structures 2014 and electrodes 111 serving as tips or ends of the deformed/collapsed limoniform structures 2014.

The electrodes 111 of the stent electrode arrays disclosed herein (e.g., the stent electrode array 2000 or the stent electrode array 2008) can be at different circumferential positions along the length of the stent body (e.g., the stent body 2002 or the stent body 2010) such that there is no overlap of electrodes 111 when the stent body is expanded in a cerebral vessel. This can advantageously ensure that the stent body has a sufficient number of electrodes 111 pointing to information rich areas of the brain upon expansion from a compressed configuration.

Any of the stents/stent electrode arrays disclosed or depicted can be wireless stents (also referred to as wireless electrode systems). The stents/stent electrode arrays can have one or more wireless transmitters. The wireless transmitters can be attached to or integrated with the stents/stent electrode arrays. The wireless transmitters can be a separate device and/or can be an arrangement of one or more electrodes 111 of the stents/stent electrode arrays. For example, an arrangement of one or more electrodes 111 can form a wireless antenna that can send and/or receive information. The electrodes 111 can record or pick up neural information and relay this information to a wireless transmitter. This recorded information can be wirelessly transmitted through the skull to a wireless receiver. The wireless receiver can decode and transmit the acquired neural information to a device such as a prosthetic limb or a visual prosthesis.

The wireless stents/stent electrode arrays can be configured for the transmission of both power and data. Power can be wirelessly transmitted to the wireless stents/stent electrode arrays to operate the circuitry of the stents/stent electrode arrays and data can be wirelessly transmitted from the wireless stents/stent electrode arrays to, for example, a control unit. In addition to or in lieu of the wireless power, the stents/stent electrode arrays can be powered with a piezoelectric energy power generator that generates energy from blood flow and/or from vascular constriction and dilation.

The wireless stents/stent electrode arrays can be fully or partially wireless. Fully wireless means that no portion of the stent/stent electrode array, including the electrodes 111 and wireless circuitry, extends beyond a vessel wall after implantation. For example, the entire stent/stent electrode array (stent and electronics) can be implanted within a cerebral vessel.

Semi-wireless means that at least a portion of the stent/stent electrode array, including electrodes 111 and/or wireless circuitry, extends beyond a vessel wall after implantation. For example, a semi-wireless stents/stent electrode array can have a wire that passes from within a cerebral vessel to outside a cerebral vessel.

As described above, the stents/stent electrode arrays can act as scaffolds for the electrodes 111 and press the electrodes 111 against a cerebral vessel wall.

In some embodiments, a system comprising such stents/stent electrode arrays can comprise between one and ten stents/stent electrode arrays implanted within a single subject/patient.

The system can have one or more stents/stent electrode arrays in wired and/or wireless communication with an implantable telemetry unit (ITU). The ITU can house a data unit that can continuously collect brain recordings. The ITU can be accessed wirelessly (e.g., via a wireless communication protocol) by the user or physician to review the neural information stored in a memory/storage unit of the ITU.

In some embodiments, the ITU can be accessed wirelessly for real-time assessment of the neural information. For example, during high-risk periods (including when the patient is exhibiting symptoms of a neural disorder or disease), the user or medical professional can access the recorded neural signals in real time. The neural data collected by the ITU can be streamed into a range of software applications that allow various real time functions. For example, the neural data collected can be communicated to third party software applications that apply software analysis of the neural data (including for seizure prediction). In this way, the collected data can be made available to third party users to generate information that can be displayed to a user/patient.

FIG. 21A illustrates a top plan view of one embodiment of an implantable electrode array 2100 in a flattened configuration. The implantable electrode array 2100 can comprise one or more shape-memory support structures 2102 and a carrier body 2104 encapsulating or partially encapsulating the one or more shape memory support structures 2102.

The implantable electrode array 2100 can further comprise a plurality of conductive traces 2106 embedded within the carrier body 2104 and extending throughout portions of the carrier body 2104. The implantable electrode array 2100 can also comprise a plurality of electrodes 2108 coupled to the carrier body 2104. Each of the conductive traces 2106 can be coupled or connected to one of the electrodes 2108. At least part of each of the electrodes 2108 can be exposed by the carrier body 2104 to allow the electrode 2108 to capture a signal such as a neural signal when the electrode array 2100 is implanted within a cerebral vessel of a subject/patient.

In some embodiments, the one or more shape-memory support structures 2102 can be made in part of a shape memory metal alloy. For example, the one or more shape-memory support structures 2102 can be made in part of a nickel-titanium alloy (e.g., Nitinol).

In other embodiments, the one or more shape-memory support structures 2102 can be made in part of a shape memory polymer. In certain embodiments, the support structures can be made of polytetrafluoroethylene (PTFE), ethylene-vinyl acetate (EVA), or polylactide (PLA).

In some embodiments, the one or more shape-memory support structures 2102 can be configured to collapse when the implantable electrode array 2100 is in a delivery configuration (for example, when the implantable electrode array 2100 is curled up or rolled up into a substantially tubular structure). In these embodiments, the one or more shape memory support structures 2102 can be configured to expand when the implantable electrode array 2100 is in a deployed or unfurled configuration (i.e., unfurled within a blood vessel or sinus vessel) to allow the carrier body 2104 to maintain vessel wall apposition.

In some embodiments, the shape memory support structures 2102 can have a substantially V-shaped or Y-shaped structure. In certain embodiments, the shape memory support structures 2102 can be connected to one another. For example, the shape memory support structures 2102 can comprise a spine that connects smaller support structures (e.g., a fishbone-like structure).

In other embodiments, the shape memory support structures 2102 can be separated from one another (see, e.g., FIG. 21A).

In some embodiments, the one or more shape memory support structures 2102 can act as a skeleton for the carrier body 2104. The carrier body 2104 can be flexible such that the entire carrier body 2104 can be folded, curled up, or rolled up to allow the implantable electrode array 2100 to fit within a small-diameter delivery catheter. The one or more shape memory support structures 2102 can expand once the implantable electrode array 2100 is deployed out of the delivery catheter and the one or more shape memory support structures 2102 can allow the carrier body 2104 to retain at least part of its enlarged, unfolded, or unrolled configuration (see, e.g., FIG. 21A).

In some embodiments, the carrier body 2104 can be made in part of a liquid crystal polymer (LCP). A liquid crystal polymer can refer to a thermoplastic polymer in which the various mesogen molecules link to form long polymeric chains.

The carrier body 2104 can be made in part of partially crystalline aromatic polyesters. For example, the LCPs can comprise semi-aromatic copolyesters, co-polyamides, polyester-co-amides, or mixtures thereof.

As shown in FIG. 21A, the carrier body 2104 can have or be defined by a digitate or finger-like structure comprising a plurality of carrier fingers 2110 or leaflets. In some embodiments, each of the carrier fingers 2110 or leaflets can comprise at least one electrode 2108.

In some embodiments, the conductive traces 2106 can be embedded within or

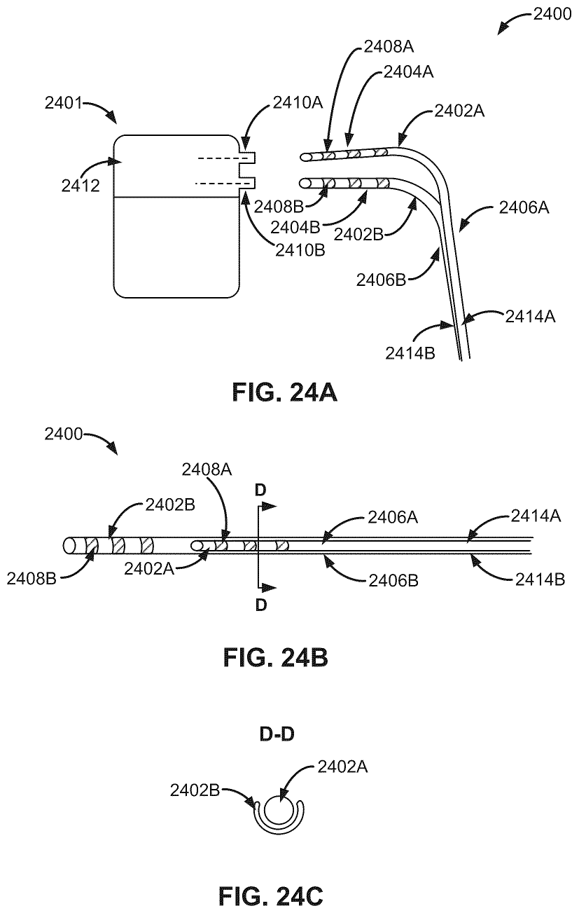

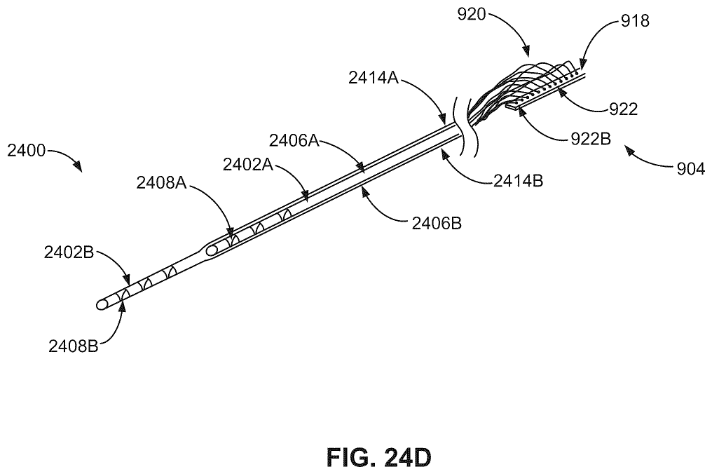

otherwise encapsulated by the carrier body 2104. The conductive traces 2106 can electrically connect the electrodes 2108 to a connector lead (see, e.g., FIG. 24A or 24B) or paddle. The paddle can be constructed similar to paddle 807 (see, e.g., FIG. 8A) or paddle 916 (see, e.g., FIG. 9) and operate in a similar manner.

In some embodiments, the conductive traces 2106 can be made in part of gold, platinum, copper, or another conductive material. In these and other embodiments, the electrodes 2108 can be made in part of gold, platinum, or another conductive material. As previously discussed, at least a portion of each of the electrodes 2108 can be exposed by the carrier body 2104 to allow the electrode 2108 to capture a signal such as a neural signal when the implantable electrode array 2100 is implanted within a neural/cerebral vessel (e.g., a neural blood vessel or sinus).

FIG. 21B illustrates an example cross-sectional view of the implantable electrode array 2100 of FIG. 21A taken along cross-section C-C. FIG. 21B illustrates that the electrically conductive traces 2106 can be layered on top of one another.

FIG. 21B also illustrates that the electrodes 2108 can be positioned on either side (e.g., a top side and a bottom side) of the carrier body 2104.

The carrier body 2104 can also serve as a sort of insulator or divider that separates the electrically conductive traces 2106 from one another and from the conductive shape-memory support structure(s) 2102.

In some embodiments, the implantable electrode array 2100 can be made in layers such that the material used to make the carrier body 2104 (e.g., the LCP) can be applied or spread out over one or more conductive traces 2106, electrodes 2108, and/or shape memory support structures 2102. This layer of the carrier body 2104 can then be heat treated or cured and another layer of the material used to make the carrier body 2104 (e.g., additional instances of the LCP) can be applied or spread out over additional instances of the conductive traces 2106, electrodes 2108, and/or shape memory support structures 2102. This can continue until all of the desired layers of the implantable electrode array 2100 are formed.

One technical problem faced by makers of implantable electrode arrays is that the substrates used to carry such electrode arrays are oftentimes conductive, which leads to the electrodes of these electrode arrays capturing poor quality signals. In addition, the conductive traces connecting such electrodes can also interfere with one another and the carriers for such traces can often only accommodate traces in a side-by-side configuration. One technical solution discovered and developed by the applicant is the implantable electrode array 2100 disclosed herein comprising one or more shape memory support structures 2102 and a monolithic carrier body 2104 encapsulating the one or more shape memory support structures 2102. Even though the shape memory support structure(s) 2102 may be conductive, these shape memory support structure(s) 2102 are encapsulated by the carrier body 2104, which is made of a flexible biocompatible dielectric material. The carrier body 2104 can robustly encapsulate the shape memory support structure(s) 2102, the conductive traces 2106, and at least part of each of the electrodes 2108. The flexible dielectric material of the carrier body 2104 can provide for good channel isolation by separating the conductive traces 2106 from one another and can reduce electrical crosstalk between neighboring conductive traces 2106.

Another technical advantage of the carrier body 2104 is that the carrier body 2104 allows for thicker conductive traces 2106. This can allow the implantable electrode array 2100 to be used for target stimulation as well as for signal recordation.

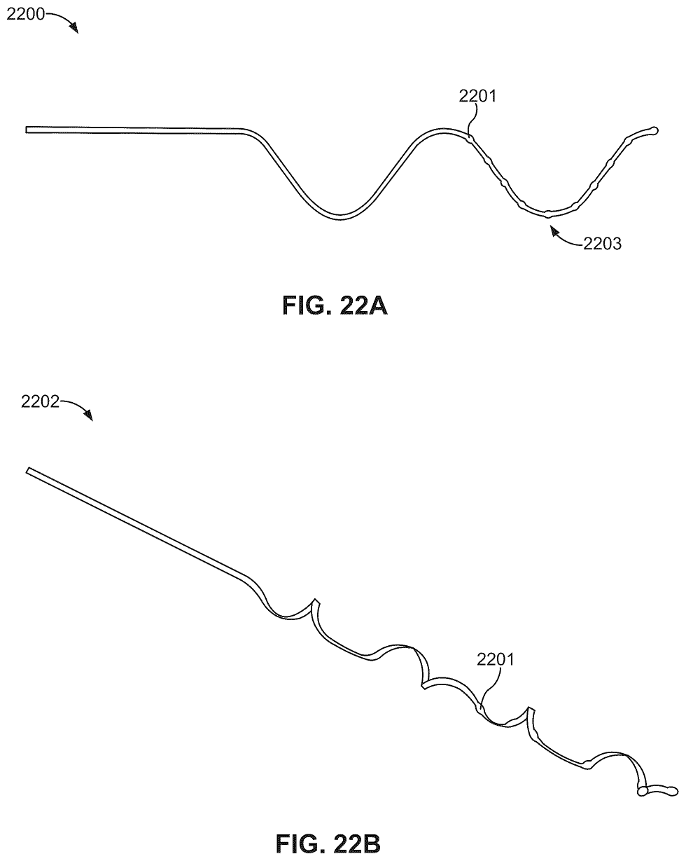

FIG. 22A illustrates one embodiment of a flexible wire 2200 comprising electrodes 2201 coupled along a length of the flexible wire 2200. The flexible wire 2200 can also have an undulating or sinusoidal wire segment 2203. As shown in FIG. 22A, at least some of the electrodes 2201 can be coupled to or otherwise positioned along the undulating or sinusoidal wire segment 2203. The flexible wire 2200 comprising the electrodes 2201 can be implanted within a cerebral vessel to record neural signals and/or stimulate neural targets. The flexible wire 2200 comprising the electrodes 2201 can also be considered an implantable electrode array.

In some embodiments, the flexible wire 2200 can be made in part of a shape-memory material such as a conductive shape-memory metallic alloy (e.g., Nitinol). The flexible wire 2200 can be delivered in a straightened configuration when housed or otherwise constrained within a delivery catheter and the undulating or sinusoidal wire segment 2203 can retain its undulating or sinusoidal shape/pattern once the flexible wire 2200 is deployed out of the delivery catheter.

FIG. 22B illustrates one embodiment of a coiled wire 2202 comprising electrodes 2201 coupled along a length of the coiled wire 2202 (for example, the electrodes 2201 can be coupled to a coiled segment of the coiled wire 2202). The coiled wire 2202 comprising the electrodes 2201 can be implanted within a small-diameter cerebral vessel to record neural signals and/or stimulate neural targets. for implantation into a cerebral vessel.

The coiled wire 2202 can be flexible and can have a thin wire diameter to access small vessels. The coiled wire 2202 can also be implanted within larger vessels depending on the size of the coils (i.e., the coil diameter). The coiled wire 2202 comprising the electrodes 2201 can also be considered an implantable electrode array.

In some embodiments, the coiled wire 2202 can be made in part of a shape-memory material such as a conductive shape-memory metallic alloy (e.g., Nitinol). The coiled wire 2202 can be delivered in a straightened configuration when housed or otherwise constrained within a delivery catheter and the coiled segment can retain its coiled/helical shape or pattern once the coiled wire 2202 is deployed out of the delivery catheter.

FIG. 23A illustrates one embodiment of a bendable wire 2300 comprising electrodes 2301 coupled along a length of the bendable wire 2300 (including along a bendable segment of the bendable wire 2300). The bendable wire 2300 comprising the electrodes 2301 can be implanted within a small-diameter cerebral vessel to record neural signals and/or stimulate neural targets. The bendable wire 2300 comprising the electrodes 2301 can also be considered an implantable electrode array.

In some embodiments, the bendable wire 2300 can be made in part of a shape-memory material such as a conductive shape-memory metallic alloy (e.g., Nitinol). The bendable wire 2300 can be delivered in a straightened configuration when housed or otherwise constrained within a delivery catheter and the bendable segment can recover or achieve its bent shape once the bendable wire 2300 is deployed out of the delivery catheter.

FIG. 23B illustrates another embodiment of a coiled wire 2302 comprising electrodes 2301 coupled along a length of the coiled wire 2302 (including along a coiled segment of the coiled wire 2302). The coiled wire 2302 comprising the electrodes 2301 can be implanted within small-diameter cerebral vessel to record neural signals and/or stimulate neural targets. The coiled wire 2302 comprising the electrodes 2301 can also be considered an implantable electrode array.

In some embodiments, the coiled wire 2302 can be made in part of a shape-memory material such as a conductive shape-memory metallic alloy (e.g., Nitinol). The coiled wire 2302 can be delivered in a straightened configuration when housed or otherwise constrained within a delivery catheter and the coiled segment can retain its coiled shape (i.e., self coil) once the coiled wire 2302 is deployed out of the delivery catheter or when a microwire is removed or retracted from the coiled wire 2302.

FIG. 24A illustrates one embodiment of a connector lead cable assembly 2400 in the process of connecting to one embodiment of an implantable receiver telemetry unit (IRTU) 2401. The connector lead cable assembly 2400 can comprise a first lead cable 2402A and a second lead cable 2402B.

The first lead cable 2402A can comprise a first lead proximal end 2404A, a first lead distal end, and a first lead intermediate segment 2406A in between the first lead proximal end 2404A and the first lead distal end. The first lead cable 2402A can also comprise a plurality of first lead connectors 2408A disposed at the first lead proximal end 2404A.

The first lead connectors 2408A can be configured to connect to or otherwise engage with electrical interfaces within a first port 2410A of the IRTU 2401. The first lead connectors 2408A can be configured to connect to or otherwise engage with the electrical interfaces within the first port 2410A when the first lead proximal end 2404A is inserted or plugged into the first port 2410A of the IRTU 2401.

The second lead cable 2402B can comprise a second lead proximal end 2404B, a second lead distal end, and a second lead intermediate segment 2406B in between the second lead proximal end 2404B and the second lead distal end. The second lead cable 2402B can also comprise a plurality of second lead connectors 2408B disposed at the second lead proximal end 2404B.

The second lead connectors 2408B can be configured to connect to or otherwise engage with electrical interfaces within a second port 2410B of the IRTU 2401. The second lead connectors 2408B can be configured to connect to or otherwise engage with the electrical interfaces within the second port 2410B when the second lead proximal end 2404B is inserted or plugged into the second port 2410B of the IRTU 2401.

Although FIG. 24A illustrates the first lead cable 2402A and the second lead cable 2402B each comprising three lead connectors, it is contemplated by this disclosure that the first lead cable 2402A can comprise between three first lead connectors 2408A and twelve first lead connectors 2408A and the second lead cable 2402B can comprise between three second lead connectors 2408B and twelve second lead connectors 2408B.

In other embodiments, the first lead cable 2402A can comprise between twelve first lead connectors 2408A and twenty first lead connectors 2408A. In these embodiments, the second lead cable 2402B can comprise between twelve second lead connectors 2408B and twenty second lead connectors 2408B.

As shown in FIG. 24A, the first port 2410A and the second port 2410B of the IRTU 2401 can be located in a header or head region 2412 of the IRTU 2401.

The first lead distal end of the first lead cable 2402A and the second lead distal end of the second lead cable 2402B can be configured to be electrically coupled or connected to an implantable electrode array (e.g., any of the implantable electrode arrays or stent electrode arrays disclosed herein).

FIG. 24B illustrates the first lead intermediate segment 2406A of the first lead cable 2402A axially stacked together with the second lead intermediate segment 2406B of the second lead cable 2402B. The first lead intermediate segment 2406A can be sized to axially stack together with the second lead intermediate segment 2406B to allow the stacked segments of the two cables to fit within a single small diameter delivery catheter (e.g., a delivery catheter with an inner lumen diameter of between about 1.42 mm to about 1.50 mm).

For example, the outer diameter of the second cable 2402B can be between about 1.20 mm to about 1.40 mm and the outer diameter of the first cable 2402A can be between about 0.75 mm to about 1.10 mm.

FIGS. 24A and 24B also illustrate that the first lead cable 2402A can comprise a first cable body 2414A and the second lead cable 2402B can comprise a second cable body 2414B. In some embodiments, the first cable body 2414A and the second cable body 2414B can be made in part of a flexible dielectric material such as a liquid crystal polymer (LCP).

The first cable body 2414A and the second cable body 2414B can also be made in part of another type of flexible dielectric or insulating material.

The first cable body 2414A can comprise a plurality of first conductive wires extending through the first cable body 2414A. The first conductive wires can connect the first lead connectors 2408A to the electrodes of one of the implantable electrode arrays or stent electrode array disclosed herein.

The second cable body 2414B can comprise a plurality of second conductive wires extending through the second cable body 2414B. The second conductive wires can connect the second lead connectors 2408B to the electrodes of one of the implantable electrode arrays or stent electrode array disclosed herein.

FIG. 24C illustrates a cross-sectional view of the first lead cable 2402A axially stacked together with the second lead cable 2402B taken along cross-section D-D of FIG. 24B.

In the embodiment shown in FIG. 24C, the second lead intermediate segment 2406B can have a substantially U-shaped cross-section. In this embodiment, the first lead intermediate segment 2406A can have a substantially circular cross-section. The first lead intermediate segment 2406A can be sized to fit within a substantially U-shaped trough of the second lead intermediate segment 2406B when the first lead intermediate segment 2406A is axially stacked together with the second lead intermediate segment 2406B.

One technical problem faced by makers of implantable electrode arrays is that the lead cables for such implantable electrode arrays often can only accommodate a limited number of connectors before the proximal segments of such lead cables become difficult to insert into a header of a telemetry unit (e.g., the IRTU). This is problematic since more connectors allow for more recording channels, which allows implantable electrode arrays to have more electrodes to capture more signals or better quality signals. One technical solution discovered and developed by the applicants is the connector lead cable assembly 2400 disclosed herein comprising a first lead cable 2402A that can axially stack together with a second lead cable 2402B. This axial stacking allows both lead cables to fit within a singular small-diameter delivery catheter. Moreover, the first lead proximal end 2404A can plug into or otherwise be inserted into a first port 2410A of a telemetry unit and the second lead proximal end 2404B can plug into or otherwise be inserted into a second port 2410B of the telemetry unit. This can allow the connector lead cable assembly 2400 to take advantage of both ports of the telemetry unit. Moreover, the distal ends of the two lead cables can merge together into a single lead body that can be coupled or connected to one implantable electrode array.

FIG. 24D illustrates that the conductive wires (e.g., the conductive wires 920) extending from the cable bodies (e.g., the first cable body 2414A, the second cable body 2414B, or a combination thereof) can be coupled to the flexible printed circuit 918 (see, also, FIG. 9). The flexible printed circuit 918 can be part of the electrical interface 904 of an implantable electrode array or stent electrode array.

The conductive wires (e.g., the conductive wires 920) can be coupled to a signal layer 1000 (see, e.g., FIG. 10) of the flexible printed circuit 918 via the second set of through-holes 922B. As previously discussed, this can provide a structural benefit as bonding the conductive wires 920 directly to a surface of a signal layer means that the bond strength depends wholly on the bonding method in the presence of a shear force.

The proximal segments of the conductive wires (e.g., the conductive wires 920) can extend through the cable bodies (e.g., the first cable body 2414A, the second cable body 2414B, or a combination thereof) and connect the first lead connectors 2408A and the second lead connectors 2408B to electrodes of an implantable electrode array via a plurality of conductive traces.

FIG. 25 illustrates certain veins and sinuses of the subject that can serve as implantation sites for the devices, systems, and electrode arrays disclosed herein.

In some embodiments, the devices, systems, and electrode arrays disclosed herein can be implanted within a venous sinus of the subject. For example, the devices, systems, and electrode arrays disclosed herein can be implanted within a superior sagittal sinus 2500, an inferior sagittal sinus 2502, a sigmoid sinus 2504, a transverse sinus 2506, or a straight sinus 2508.

In other embodiments, the devices, systems, and electrode arrays disclosed herein can be implanted within a superficial cerebral vein of the subject. For example, the tubular member 101 carrying the electrode array 900 can be implanted within at least one of a vein of Labbe 2510, a vein of Trolard 2512, a Sylvian vein 2514, and a Rolandic vein 2516.

The devices, systems, and electrode arrays disclosed herein can also be implanted within a deep cerebral vein of the subject. For example, the tubular member 101 carrying the electrode array 990 can be implanted within at least one of a vein of Rosenthal 2518, a vein of Galen 2520, a superior thalamostriate vein 2522, an inferior thalamostriate vein, and an internal cerebral vein 2524.

In further embodiments, the devices, systems, and electrode arrays disclosed herein can also be implanted within at least one of a central sulcal vein, a post-central sulcal vein, and a pre-central sulcal vein. In additional embodiments, the devices, systems, and electrode arrays disclosed herein can also be implanted or configured to be implanted within a vessel extending through a hippocampus or amygdala of the subject.

Once implanted, the devices, systems, and electrode arrays disclosed herein can be configured to detect or record an electrophysiological signal of the subject. In some embodiments, the electrophysiological signal can be a local field potential (LFP) and/or an intracranial/cortical EEG measured within a cerebral or cortical vessel (e.g., a venous sinus or cortical vein). In other embodiments, the electrophysiological signal can be an electrocorticography (ECoG) signal. In some embodiments, the electrophysiological signal can be a transitory neural oscillatory burst.

As previously discussed, when the intracorporeal target to be stimulated by the devices, systems, and electrode arrays disclosed herein is a vagus nerve of the subject, the devices, systems, and electrode arrays disclosed herein can be implanted within an internal jugular vein (either a right internal jugular vein 2526 or a left internal jugular vein 2528) or an internal carotid artery.

In other embodiments, the intracorporeal target or stimulation target can be the cerebellum 2530 of the subject. In these embodiments, the devices, systems, and electrode arrays disclosed herein can be implanted within at least one of a sigmoid sinus 2504 and a straight sinus 2508 of the subject. Moreover, the devices, systems, and electrode arrays disclosed herein can also be implanted within a transverse sinus 2506 of the subject. At least part of the cerebellum 2530 is adjacent to the sigmoid sinus 2504, the straight sinus 2508, and the transverse sinus 2506.

In some embodiments, stimulating the intracorporeal target or the stimulation target via the devices, systems, and electrode arrays disclosed herein can increase blood flow to the intracorporeal target or raise levels of certain neurotransmitters involved in suppressing seizure activity. Moreover, stimulating the intracorporeal target via the devices, systems, and electrode arrays disclosed herein can also lead to sodium-channel inactivation (using high-frequency stimulation), long-term depression of certain neurotransmitters (using high-frequency stimulation), and/or glutamatergic depression (using both low-frequency and high-frequency stimulation).

For example, when stimulating cortical or cerebral targets, the electrical impulse can be bipolar with the voltage of the electrical impulse increased from 1V to 7 V in 0.25 V steps. The electrical impulse generated can have a pulse width of between 90 uS to about 540 uS, a frequency between about 3 Hz to 5 Hz in a low-frequency range, and a frequency between about 50 Hz to 130 Hz in a high-frequency range.

A number of embodiments have been described. Nevertheless, it will be understood by one of ordinary skill in the art that various changes and modifications can be made to this disclosure without departing from the spirit and scope of the embodiments. Elements of systems, devices, apparatus, and methods shown with any embodiment are exemplary for the specific embodiment and can be used in combination or otherwise on other embodiments within this disclosure. For example, the steps of any methods depicted in the figures or described in this disclosure do not require the particular order or sequential order shown or described to achieve the desired results. In addition, other steps or operations may be provided, or steps or operations may be eliminated or omitted from the described methods or processes to achieve the desired results. Moreover, any components or parts of any apparatus or systems described in this disclosure or depicted in the figures may be removed, eliminated, or omitted to achieve the desired results. In addition, certain components or parts of the systems, devices, or apparatus shown or described herein have been omitted for the sake of succinctness and clarity.

Accordingly, other embodiments are within the scope of the following claims and the specification and/or drawings may be regarded in an illustrative rather than a restrictive sense.

Each of the individual variations or embodiments described and illustrated herein has discrete components and features which may be readily separated from or combined with the features of any of the other variations or embodiments. Modifications may be made to adapt a particular situation, material, composition of matter, process, process act(s) or step(s) to the objective(s), spirit, or scope of the present invention.

Methods recited herein may be carried out in any order of the recited events that is logically possible, as well as the recited order of events. Moreover, additional steps or operations may be provided or steps or operations may be eliminated to achieve the desired result.

Furthermore, where a range of values is provided, every intervening value between the upper and lower limit of that range and any other stated or intervening value in that stated range is encompassed within the invention. Also, any optional feature of the inventive variations described may be set forth and claimed independently, or in combination with any one or more of the features described herein. For example, a description of a range from 1 to 5 should be considered to have disclosed subranges such as from 1 to 3, from 1 to 4, from 2 to 4, from 2 to 5, from 3 to 5, etc. as well as individual numbers within that range, for example 1.5, 2.5, etc. and any whole or partial increments therebetween.

All existing subject matter mentioned herein (e.g., publications, patents, patent applications) is incorporated by reference herein in its entirety except insofar as the subject matter may conflict with that of the present invention (in which case what is present herein shall prevail). The referenced items are provided solely for their disclosure prior to the filing date of the present application. Nothing herein is to be construed as an admission that the present invention is not entitled to antedate such material by virtue of prior invention.

Reference to a singular item, includes the possibility that there are plural of the same items present. More specifically, as used herein and in the appended claims, the singular forms “a,” “an,” “said” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation. Unless defined otherwise, all technical and scientific terms used herein have the same meaning as commonly understood by one of ordinary skill in the art to which this invention belongs.

Reference to the phrase “at least one of”, when such phrase modifies a plurality of items or components (or an enumerated list of items or components) means any combination of one or more of those items or components. For example, the phrase “at least one of A, B, and C” means: (i) A; (ii) B; (iii) C; (iv) A, B, and C; (v) A and B; (vi) B and C; or (vii) A and C.

In understanding the scope of the present disclosure, the term “comprising” and its derivatives, as used herein, are intended to be open-ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives. Also, the terms “part,” “section,” “portion,” “member” “element,” or “component” when used in the singular can have the dual meaning of a single part or a plurality of parts. As used herein, the following directional terms “forward, rearward, above, downward, vertical, horizontal, below, transverse, laterally, and vertically” as well as any other similar directional terms refer to those positions of a device or piece of equipment or those directions of the device or piece of equipment being translated or moved.

Finally, terms of degree such as “substantially”, “about” and “approximately” as used herein mean the specified value or the specified value and a reasonable amount of deviation from the specified value (e.g., a deviation of up to +0.1%, +1%, +5%, or +10%, as such variations are appropriate) such that the end result is not significantly or materially changed. For example, “about 1.0 cm” can be interpreted to mean “1.0 cm” or between “0.9 cm and 1.1 cm.” When terms of degree such as “about” or “approximately” are used to refer to numbers or values that are part of a range, the term can be used to modify both the minimum and maximum numbers or values.

It will be understood by one of ordinary skill in the art that the various methods disclosed herein may be embodied in a non-transitory readable medium, machine-readable medium, and/or a machine accessible medium comprising instructions compatible, readable, and/or executable by a processor or server processor of a machine, device, or computing device. The structures and modules in the figures may be shown as distinct and communicating with only a few specific structures and not others. The structures may be merged with each other, may perform overlapping functions, and may communicate with other structures not shown to be connected in the figures. Accordingly, the specification and/or drawings may be regarded in an illustrative rather than a restrictive sense.

This disclosure is not intended to be limited to the scope of the particular forms set forth, but is intended to cover alternatives, modifications, and equivalents of the variations or embodiments described herein. Further, the scope of the disclosure fully encompasses other variations or embodiments that may become obvious to those skilled in the art in view of this disclosure.

1. A system for implanting electrodes within a cerebral vessel of a subject, comprising:
a microwire; and
a tubular member comprising:
a tubular body sized to fit within the cerebral vessel;
a central lumen extending through the tubular body, wherein the central lumen is sized to be advanced over the microwire,
a plurality of peripheral lumens extending through the tubular body, wherein the plurality of peripheral lumens surround the central lumen,
a plurality of wires extending through each of the peripheral lumens; and
a plurality of electrodes coupled to the tubular body, wherein the plurality of electrodes are spaced apart along a length of the tubular body, and wherein a conductive portion of one of the wires is connected to one of the electrodes.

2. (canceled)

3. (canceled)

4. (canceled)

5. (canceled)

6. (canceled)

7. (canceled)

8. (canceled)

9. (canceled)

10. (canceled)

11. (canceled)

12. (canceled)

13. (canceled)

14. (canceled)

15. (canceled)

16. (canceled)

17. (canceled)

18. (canceled)

19. (canceled)

20. (canceled)

21. The system of claim 1, further comprising a pre-shaped helical wire sized to extend through the central lumen of the tubular body when the microwire is removed from the central lumen, wherein the pre-shaped helical wire is configured to force at least part of the tubular body to attain a helical or coiled configuration when the pre-shaped helical wire is extended through at least part of the central lumen.

22. The system of claim 1, wherein the tubular body is configured to self-coil into a helical or coiled configuration in response to the microwire being removed from the central lumen of the tubular member, and wherein the plurality of electrodes are configured to press against a vessel wall of the cerebral vessel when the tubular body is in the helical or coiled configuration.

23. The system of claim 1, further comprising an anchoring wire comprising a hooked portion at a distal end of the anchoring wire, wherein the anchoring wire is sized to extend through the central lumen of the tubular body when the microwire is removed from the central lumen, and wherein the hooked portion of the anchoring wire is configured to extend distally out of the central lumen to secure the tubular member within the cerebral vessel.

24. The system of claim 1, further comprising an anchoring wire comprising:
a wire distal end,
an anchoring segment, and
a proximal wire segment located proximal to the anchoring segment,
wherein the anchoring wire is sized to extend through one of the peripheral lumens or another lumen extending through the tubular body,
wherein the wire distal end of the anchoring wire is coupled to a distal point along a tubular body wall of the tubular member,
wherein at least part of the anchoring segment of the anchoring wire is configured to extend through an opening defined along the tubular body wall of the tubular body, wherein the opening is positioned proximal to the distal point, and
wherein at least part of the anchoring segment is configured to form a looped segment outside of the tubular body when part of the proximal wire segment is advanced distally, and wherein the looped segment is configured to secure the tubular member within the cerebral vessel.

25. The system of claim 1, wherein at least one of the electrodes comprises an electrode body converging at a sharp point to enable the electrode to penetrate through a vessel wall of the cerebral vessel when the electrode is pressed against the vessel wall.

26. The system of claim 25, wherein the electrode body houses a medicament or pharmaceutical, and wherein the electrode body is configured to release the medicament or pharmaceutical when the electrode is pressed against the vessel wall.

27. (canceled)

28. (canceled)

29. (canceled)

30. The system of claim 1, wherein the plurality of electrodes comprises a first electrode and a second electrode, wherein the first electrode comprises a first electrode body converging at a first sharp point to enable the first electrode to penetrate through a vessel wall of the cerebral vessel when the first electrode is pressed against the vessel wall, wherein the second electrode comprises a second electrode body converging at a second sharp point to enable the second electrode to penetrate through the vessel wall of the cerebral vessel when the second electrode is pressed against the vessel wall.

31. The system of claim 30, wherein the first electrode has a first electrode height, wherein the second electrode has a second electrode height, and wherein the first electrode height is greater than the second electrode height.

32. (canceled)

33. (canceled)

34. (canceled)

35. (canceled)

36. A device for implantation within a cerebral vessel of a subject, comprising:
a tubular body sized to fit within the cerebral vessel;
a central lumen extending through the tubular body;
a plurality of peripheral lumens extending through the tubular body, wherein the plurality of peripheral lumens surround the central lumen;
a plurality of wires extending through each of the peripheral lumens; and
a plurality of electrodes coupled to the tubular body, wherein the plurality electrodes are spaced apart along a length of the tubular body, and wherein a conductive portion of one of the wires is connected to one of the electrodes.

37. A device for implantation within a cerebral vessel of a subject, comprising:
a tubular body sized to fit within the cerebral vessel;
a plurality of lumens extending through the tubular body;
a plurality of wires extending through each of the lumens; and
a plurality of electrodes coupled to the tubular body, wherein the plurality electrodes are spaced apart along a length of the tubular body, and wherein a conductive portion of one of the wires is connected to one of the electrodes.

38. (canceled)

39. (canceled)

40. (canceled)

41. (canceled)

42. (canceled)

43. (canceled)

44. (canceled)

45. (canceled)

46. (canceled)

47. (canceled)

48. (canceled)

49. (canceled)

50. (canceled)

51. (canceled)

52. (canceled)

53. (canceled)

54. (canceled)

55. (canceled)

56. (canceled)

57. (canceled)

58. (canceled)

59. (canceled)

60. (canceled)

61. (canceled)

62. (canceled)

63. (canceled)

64. (canceled)

65. (canceled)

66. A method of implanting electrodes within a cerebral vessel of a subject, comprising:
delivering a microwire to a target site in a cerebral vessel;
advancing a tubular member over the microwire to the target site, wherein the tubular member comprises:
a tubular body;
a central lumen extending through the tubular body, wherein the central lumen is sized to be advanced over the microwire,
a plurality of peripheral lumens extending through the tubular body, wherein the plurality of peripheral lumens surround the central lumen,
a plurality of wires extending through each of the peripheral lumens, and
a plurality of electrodes coupled to the tubular body, wherein the plurality electrodes are spaced apart along a length of the tubular body, and wherein a conductive portion of each of the wires is connected to one of the electrodes; and
removing the microwire when the tubular member is secured at the target site.

67. (canceled)

68. (canceled)

69. (canceled)

70. (canceled)

71. (canceled)

72. (canceled)

73. (canceled)

74. (canceled)

75. (canceled)

76. (canceled)

77. (canceled)

78. A stent, comprising:
a stent body comprising a plurality of struts coupled together with strut crosslinks; and
a plurality of electrodes coupled along the stent body,
wherein one of the struts comprises:
a base layer made in part of a shape memory alloy,
a first insulating layer disposed on top of at least part of the base layer,
a first conductive track disposed on top of at least part of the first insulating layer, wherein the first conductive track is electrically coupled to one of the electrodes,
a second insulating layer disposed on top of at least part of the first conductive track,
a second conductive track disposed on top of at least part of the second insulating layer, wherein the second conductive track is electrically coupled to another one of the electrodes, and
a third insulating layer disposed on top of at least part of the second conductive track.

79. (canceled)

80. (canceled)

81. (canceled)

82. (canceled)

83. (canceled)

84. (canceled)

85. (canceled)

86. (canceled)

87. (canceled)

88. (canceled)

89. (canceled)

90. (canceled)

91. A device for implanting electrodes within a cerebral vessel of a subject, comprising:
a tubular member comprising:
a tubular body sized to fit within the cerebral vessel and a body distal end, and
a lumen extending through the tubular body,
a plurality of wires extending through the lumen, wherein each of the wires comprises a distal wire segment and a distal wire end serving as a distal terminus of the wire, wherein the distal wire segment of each of the wires protrude out of the lumen beyond the body distal end; and
an electrode coupled to the distal wire end of each of the wires.

92. (canceled)

93. (canceled)

94. (canceled)

95. A method of delivering a neurovascular device within a patient, the method comprising:
pre-setting a curved section of a lead device at a predetermined dimension and a predetermined shape, wherein the curved section comprises one or more electrodes;
advancing the lead device via a catheter and over a guidewire to a target location in a cerebral vessel, wherein the curved section straightens out via material stress during advancement;
advancing the lead device out of the catheter once the catheter is at the target location, wherein the curved section deforms to its predetermined dimension and predetermined shape to produce an outward radial force on the cerebral vessel to anchor the lead device in place; and
stimulating the cerebral vessel with the one or more electrodes.

96. An implantable electrode array, comprising:
an implantable carrier, comprising:
a carrier body comprising a carrier proximal end and a carrier distal end,
a plurality of electrodes coupled to the carrier body, and
a paddle extending from the carrier proximal end of the carrier body;
a flexible printed circuit coupled to the paddle of the implantable carrier; and
one or more conductive wires coupled to the flexible printed circuit.

97. (canceled)

98. (canceled)

99. (canceled)

100. (canceled)

101. (canceled)

102. (canceled)

103. (canceled)

104. (canceled)

105. (canceled)

106. (canceled)

107. (canceled)

108. (canceled)

109. (canceled)

110. (canceled)

111. (canceled)

112. (canceled)

113. (canceled)

114. (canceled)

115. (canceled)

116. An electrical interface for an implantable electrode array, comprising:
a flexible printed circuit configured to be coupled to an implantable carrier,
wherein the flexible printed circuit comprises a plurality of layers stacked on top of one another, and
wherein the flexible printed circuit comprises a first set of through-holes and a second set of through-holes, wherein the first set of through-holes are positioned distal to the second set of through-holes, wherein at least one through-hole of the first set of through-holes is electrically coupled to at least one through-hole of the second set of through-holes via conductive traces disposed on one of the plurality of layers; and
one or more conductive wires coupled to the flexible printed circuit.

117. (canceled)

118. (canceled)

119. (canceled)

120. (canceled)

121. (canceled)

122. (canceled)

123. (canceled)

124. (canceled)

125. (canceled)

126. (canceled)

127. (canceled)

128. (canceled)

129. (canceled)

130. (canceled)

131. An implantable electrode array, comprising:
one or more shape memory support structures;
a carrier body encapsulating the one or more shape memory support structures;
a plurality of conductive traces embedded within the carrier body; and
a plurality of electrodes coupled to the carrier body, wherein each of the conductive traces is connected to one of the electrodes, and wherein at least part of each of the electrodes is exposed by the carrier body to allow the electrode to capture a signal.

132. (canceled)

133. (canceled)

134. (canceled)

135. (canceled)

136. (canceled)

137. (canceled)

138. (canceled)

139. (canceled)

140. (canceled)

141. A connector lead cable assembly for an implantable electrode array, comprising:
a first lead cable comprising a first lead proximal end, a first lead distal end, and a first lead intermediate segment in between the first lead proximal end and the first lead distal end;
a second lead cable comprising a second lead proximal end, a second lead distal end, and a second lead intermediate segment in between the second lead proximal end and the second lead distal end;
wherein the first lead cable further comprises a plurality of first lead connectors disposed at the first lead proximal end for connecting to electrical interfaces within a first port of an implantable receiver telemetry unit (IRTU),
wherein the second lead cable further comprises a plurality of second lead connectors disposed at the second lead proximal end for connecting to electrical interfaces within a second port of the IRTU,
wherein the first lead distal end and the second lead distal end are configured to be coupled to the implantable electrode array, and
wherein the first lead intermediate segment is configured to axially stack together with the second lead intermediate segment such that the first lead intermediate segment stacked together with the second lead intermediate segment fit within a single small diameter delivery catheter.

142. (canceled)

143. (canceled)

144. (canceled)

145. (canceled)

146. A system for implanting electrodes within a cerebral vessel of a subject, comprising:
an outer tubular member comprising an outer tubular body, a plurality of holes defined along the outer tubular body, and an outer hollow lumen extending through an interior of the outer tubular body; and
an inner tubular member comprising an inner tubular body and a plurality of electrodes extending laterally or orthogonally from the inner tubular body,
wherein at least part of the inner tubular member is configured to fit within the outer hollow lumen and be detachably coupled to the outer tubular member via a threaded connection when the system is in an assembled configuration,
wherein the plurality of electrodes are configured to be housed or constrained within the outer tubular member when the system is being delivered to an implantation site, and
wherein the outer tubular member and the inner tubular member are configured to rotate with respect one another to align the plurality of electrodes with the plurality of holes to expose the plurality of electrodes or allow the plurality of electrodes to extend through the plurality of holes when the system is positioned at the implantation site.
